(19) United States
(12) Patent Application Publication
(10) Pub. No.: US 2022/0152048 A1
(43) Pub. Date: May 19, 2022

(54) Botanical Extracts and Compounds from Castanea Plants and Methods of Use

(71) Applicant: Emory University, Atlanta, GA (US)

(72) Inventors: Cassandra L. Quave, Atlanta, GA (US); James Lyles, Sandy Springs, GA (US); Gina Porras-Brenes, Atlanta, GA (US); Akram Salam, Clarkston, GA (US)

(21) Appl. No.: 17/435,594

(22) PCT Filed: Feb. 28, 2020
(86) PCT No.: PCT/US2020/020335

§ 371 Date: Sep. 1, 2021

Related U.S. Application Data
(60) Provisional application No. 62/812,850, filed on Mar. 1, 2019.
(60) Provisional application No. 62/873,461, filed on Jul. 12, 2019.

(51) Int. Cl. A61K 31/567; A61K 45/06

(52) U.S. Cl. CPC A61K 31/567; A61K 45/06

(57) ABSTRACT

This disclosure relates to extracts from chestnut plants and compositions comprising compounds contained therein. In certain embodiments, the extracts are derived from the leaves of a Castanea plant. In certain embodiments, the disclosure relates to methods of treating or preventing bacterial infections, acne, and other related uses.

### CROSS-REFERENCE TO RELATED APPLICATIONS

This application claims the benefit of U.S. Provisional Application No. 62/812,850 filed Mar. 1, 2019 and U.S. Provisional Application No. 62/873,461 filed Jul. 12, 2019. The entirety of each of these applications is hereby incorporated by reference for all purposes.

### BACKGROUND

Since the widespread introduction of antibiotics in the 1940s, the same storyline has repeated itself over and over again: a new antibiotic is introduced, and then resistant variants emerge and quickly spread, effectively limiting the utility and lifespan of the drug. From an evolutionary biology perspective, this is not surprising; indeed, resistant mutants are expected to arise when any lifeform with the ability to rapidly reproduce and mutate is faced with a direct selective pressure, especially when a single drug is used against a single target. Staphylococci are frequently the cause of hospital infections such as infections from implanted medical devices. Many Staphylococci strains have become resistant to many modern-day antibiotics. Improved therapies are needed.

One proposed strategy to overcome the problem of resistant variants is to indirectly attack bacteria by interfering with their means of communication, also known as quorum sensing. Targeting microbial communication makes sense because bacteria coordinate many of their virulence and pathogenesis pathways through these systems. Quave et al., report quorum sensing inhibitors of Staphylococcus aureus from botanical extracts. Planta Med. 2011, 77(02):188-95. See also U.S. Pat. No. 10,195,241.

Castanea sativa (chestnut) is a flowering plant in the family Fagaceae which can be found in Europe. See Braga et al., Nat Prod Res., 2015, 29(1):1-18. Almeida et al. report in vivo skin irritation potential of a Castanea sativa (Chestnut) leaf extract. Basic & Clinical Pharmacol Toxicol, 2008, 103(5):461-7. See also Almeida et al. J Photochem Photobiol B: Biol, 2015, 144(0):28-34. Henry et al. report cosmetic compositions containing an extract of leaves of the Castanea sativa plant and cosmetic treatments. U.S. Pat. No. 8,067,044 (2011).

Garo et al., report asiatic acid and corosolic acid enhance the susceptibility of Pseudomonas aeruginosa biofilms to tobramycin. Antimicrob Agents Chemother, 2007, 51(5):1813-7. See also Rangasamy et al. South African J Botany, 2014, 93:198-203.

Wong et al. report aqueous methanolic extracts of Melastoma malabathricum L. exhibited antibacterial activity. Nat Prod Res, 2012,26(7):609-18

Perioni et al. report a survey on the natural ingredients used in folk cosmetics, cosmeceuticals and remedies for healing skin diseases. J Ethnopharmacol, 2004, 91(2-3):331-44.

References cited herein are not an admission of prior art.

### SUMMARY

This disclosure relates to extracts from chestnut plants and compositions comprising one or more compounds contained therein and related uses reported herein. In certain embodiments, the extracts are derived from the leaves of a Castanea plant such as Castanea sativa.

In certain embodiments, the disclosure relates to extracts comprising a leaf derived mixture of compounds from a Castanea plant wherein the extracting process comprises one or more of the following steps of: mixing a leaf with methanol under conditions such that leaf compounds dissolves in the methanol and removing the methanol providing a methanol derived mixture of compounds; partitioning the methanol derived mixture of compounds in hexane and water providing a water derived mixture of compounds; partitioning the water derived mixture of compounds by mixing the water with ethyl acetate under conditions such that leaf compounds dissolve in the ethyl acetate and removing the ethyl acetate providing an ethyl acetate derived mixture of compounds; and purifying the ethyl acetate derived mixture of compounds by liquid chromatography through silica with a mobile phase comprising hexane and ethyl acetate; wherein the mobile phase comprises increasing amounts of ethyl acetate, and a mobile phase fraction is isolated comprising a leaf derived mixture of compounds.

In certain embodiments, contemplated compositions include compounds further separated from impurities. In certain embodiment, this disclosure contemplates that compounds disclosed herein are used in a substantially purified form. For example, prior to addition to a pharmaceutical formulation the compounds may be purified to contain less than 50%, 40%, 30%, 20%, 10%, or 5%, by weight of impurities or derivatives of the compounds. In certain embodiments, contemplated compositions include racemic mixtures and compounds in of greater than 5%, 10%, 20%, 40%, 80%, or 95% enantiomeric excess.

In certain embodiments, contemplated compounds are

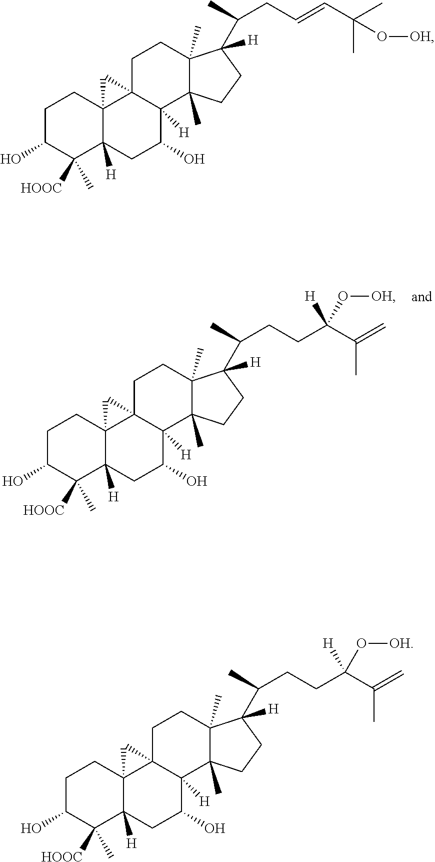 [SMILES: C[C@@H](C/C=C/C(C)(C)OO)[C@@H]1CC[C@]2(C)[C@H]3[C@H](O)C[C@@H]4[C@@](C)(C(=O)O)[C@H](O)CC[C@]45C[C@]35CC[C@@]12C ; C=C(C)[C@H](CC[C@H](C)[C@@H]1CC[C@]2(C)[C@H]3[C@H](O)C[C@@H]4[C@@](C)(C(=O)O)[C@H](O)CC[C@]45C[C@]35CC[C@@]12C)OO ; C=C(C)[C@@H](CC[C@H](C)[C@@H]1CC[C@]2(C)[C@H]3[C@H](O)C[C@@H]4[C@@](C)(C(=O)O)[C@H](O)CC[C@]45C[C@]35CC[C@@]12C)OO]

In certain embodiments, this disclosure relates to methods of treating or preventing a bacterial infections or acne comprising administering to a subject in need thereof or contacting the skin of a subject in need thereof with a formula comprising an extract or one or more compounds in an extract as disclosed herein. In certain embodiments, the formula is administered in combination with another antibiotic.

In certain embodiments, this disclosure relates to methods of treating or preventing a toxin-mediated bacterial infection comprising administering an effective amount of a Castanea extract or compounds contained therein to a subject in need thereof, including a subject at risk of, exhibiting symptoms of, or diagnosed with a staphylococcal scalded skin syndrome (esp. in neonates), abscesses, necrotizing fasciitis, sepsis, or atopic dermatitis (eczema).

In certain embodiments, the subject is at risk of, exhibiting symptoms of, or diagnosed with toxic shock syndrome, scalded skin syndrome, abscesses, furuncles, cellulitis, folliculitis, bloodstream infections, medical device infections, pneumonia, osteomyelitis, staphylococcal food poisoning, skin and soft tissue infections, endocarditis, eczema, atopic dermatitis, psoriasis, impetigo, septic arthritis, brain abscess, burn wounds, venous ulcers, diabetic foot ulcers, surgical wounds, post-operation infections, carbuncles, meningitis, bacteremia, necrotizing pneumonia, or necrotizing fasciitis.

In certain embodiments, the disclosure contemplates the use of an extract or one or more compounds in an extract disclosed herein in a tampon for the treatment or prevention of toxic shock syndrome.

In certain embodiments, the disclosure relates to a pharmaceutical or cosmetic formulation comprising an extract or one or more compounds in an extract disclosed herein and a pharmaceutically acceptable excipient or cosmetically acceptable excipient. In certain embodiments, the disclosure relates to a liquid or gel formulation optionally further comprising an antibacterial agent, a topical steroid, an anti-inflammatory agent, a promoter of skin barrier function, a skin moisturizer or combinations thereof. In certain embodiments the antibacterial agent is daptomycin, linezolid, vancomycin, nafcillin, cefazolin, dicloxacillin, clindamycin, rifampin, sulfamethoxazole-trimethoprim (Bactrim), or botanical antibacterial agents, e.g., Melaleuca alternifolia tea tree oil.

In certain embodiments, the compound is in the form of an aqueous solution further comprising a buffering agent, oil, phosphate buffer, sodium or potassium salt, a saccharide, polysaccharide, or solubilizing agent.

Uses as an injectable product (for intravenous, intramuscular, subcutaneous, intradermal injections, intraperitoneal, or other administration) are contemplated. In certain embodiments, the disclosure relates to a pharmaceutical injectable formulation comprising an extract or one or more compounds in an extract disclosed herein and a pharmaceutically acceptable excipient. In certain embodiments, the disclosure relates to an injectable formulation optionally further comprising an antibacterial agent, a topical steroid, an anti-inflammatory agent, or combinations thereof. In certain embodiments the antibacterial agent is daptomycin, linezolid, vancomycin, nafcillin, cefazolin, dicloxacillin, clindamycin, rifampin, sulfamethoxazole-trimethoprim (Bactrim), or botanical antibacterial agents, e.g., Melaleuca alternifolia tea tree oil.

In certain embodiments, the disclosure relates to a pharmaceutical composition comprising an extract or one or more compounds in an extract disclosed herein formulated with an enteric coating.

In certain embodiments, the disclosure relates to a solid or liquid soap or lotion comprising an extract or one or more compounds in an extract disclosed herein and a fatty acid.

In certain embodiments, the disclosure relates to a medical device comprising a coating comprising an extract or one or more compounds in an extract disclosed herein.

In certain embodiments, the disclosure relates to a tampon or tampon fibers comprising an extract or one or more compounds in an extract disclosed herein and an absorbent material.

In certain embodiments, the disclosure relates to a wound dressings or wound rinse comprising an extract or one or more compounds in an extract disclosed herein wherein the wound dressing comprises an absorbent pad and optionally an adhesive.

In certain embodiments, the disclosure relates to a disinfectant spray or wipe formulation for surfaces and fomites, comprising an extract and one or one or more compounds in an extract disclosed herein wherein the spray or wipe comprises an extract or one or more compounds in an extract disclosed herein such as a formula including chlorine-based disinfectants.

### BRIEF DESCRIPTION OF THE DRAWINGS

FIG. 1 illustrates a schematic of the Staphylococcus aureus accessory gene regulator system. The agr locus has been investigated in detail and is known to contain two divergent transcripts named RNAII and RNAIII The RNAII transcript is an operon of four genes, agrBDCA, that encode factors required to synthesize AIP and activate the regulatory cascade. Briefly, AgrD is the precursor peptide of AIP, AgrB is a membrane protease involved in generating AIP, AgrC is a histidine kinase that is activated by binding AIP, and AgrA is a response regulator that induces transcription of both RNAII and RNAIII. The RNAIII transcript yields a regulatory RNA molecule that acts as the primary effector of the agr system by up-regulating extracellular virulence factors and down-regulating cell surface proteins. The agr pathway is illustrated here with potential target sites for 224C-F2.

FIG. 2A illustrates Castanea sativa methanolic leaf extract (224) fractionation scheme.

FIG. 2B shows flash chromatography of 224C utilized 12 g of material per run. A silica column was used with three mobile phases: hexanes, ethyl acetate, methanol. The horizontal double-headed arrows represent the elution times of each fractions. Vertical lines highlight the elution time of 224C-F2c. The chromatograms at several wavelengths are shown.

FIG. 2C illustrates the fractionation scheme of 224C-F2c via preparative HPLC. Each fraction is a numbered PF. The fractionation scheme of 224C-F2c resulted in the collection of fractions. Injections into the preparative HPLC were of 2000 μL at 25 mg/mL. A C-18 column was used with two mobile phases: [A] 0.1% formic acid in water, [B] 0.1% formic acid in acetonitrile. The vertical lines denoted by ‘42’, represent the elution time of PF42, which occurs from 35.0-35.5 min. The chromatogram at 314 nm is shown.

FIG. 2D illustrates the fractionation scheme of 224C-F2c-PF42 via preparative HPLC. The fractionation scheme of 224C-F2c-PF42 resulted in the collection of thirty-two fractions. Injections into the preparative HPLC were of 2000 μL at 25 mg/mL. A C-18 column was used with two mobile phases: [A] 0.1% formic acid in water, [B] 0.1% formic acid in acetonitrile. The fraction that elutes from 2.5-3.0 min denoted by ‘6’ and 3.0-3.5 min denoted by ‘7’ is SF7. The chromatogram at 217 nm is shown.

FIG. 2E illustrates the proposed structures of compounds 1a, 1b, and 2 in 224C-F2c-PF42-SF6 and 224C-F2c-PF42-SF7.

FIG. 2F illustrates the X-ray crystal structure of compound 1a derived from the 224C-F2c-PF42-SF6 as illustrated in FIG. 2E.

FIG. 2G illustrates the X-ray crystal structure of compound 2 derived from the 224C-F2c-PF42-SF7 faction as illustrated in FIG. 2E.

FIG. 3 shows activity data of 224C-F2c-PF42-SF6 and 224C-F2c-PF42-SF7 fractions tested in a Staphylococcus aureus reporter strain of agr I expression. Strain AH1677 was treated with a range of doses of SF6, SF7, and a combination of the two. OD refers to growth, while FLD refers to fluorescence of the gene reporter. Activity of SF6 and SF7 is slightly reduced as compared to the parent fraction and positive control, 224C-F2c. The combined effects of SF6 and SF7 are additive; a dose 4 μg/mL SF6+4 μg/mL SF7 has the same effect as an 8 μg/mL dose of either.

FIG. 4A illustrates additional compounds contemplated by this disclosure.

FIG. 4B illustrates additional compounds contemplated by this disclosure.

FIG. 5 illustrates a rearrangement showing how 1a and 1b can potentially interchange where both are present in fraction SF6.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used herein, the term “combination with” when used to describe administration with an additional treatment means that the agent may be administered prior to, together with, or after the additional treatment, or a combination thereof.

As used herein, “subject” refers to any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, “relative abundance” refers to amount determined from electrospray ionization mass spectrometry. Mass spectrometry is an analytical technique that can provide both qualitative (structure) and quantitative (molecular mass or concentration) information on analyte molecules after their conversion to ions. The molecules of interest are first introduced into the ionization source of the mass spectrometer, where they are first ionized to acquire positive or negative charges. The ions then travel through the mass analyzer and arrive at different parts of the detector according to their mass/charge (m/z) ratio. After the ions contact the detector, useable signals are generated and recorded by a computer system. The computer displays the signals graphically as a mass spectrum showing the relative abundance of the signals according to their m/z ratio.

Chestnut Leaf Extracts Block Staphylococcus aureus Virulence and Pathogenesis

Quorum quenching activity has been discovered in the natural products extracted from Castanea sativa leaves. The extract is able to attenuate virulence by quenching S. aureus agr-mediated quorum sensing, effectively blocking production of harmful exotoxins at sub-inhibitory concentrations for growth. Experiments indicate a lack of cytotoxicity to human skin cells, lack of growth inhibitory activity against the normal skin microflora, lack of resistance development, and efficacy in a skin abscess animal model.

Staphylococcus aureus is an abundant, opportunistic pathogen that is the causative agent of numerous infections. Due to its prevalence as a leading cause of healthcare-associated infection, and its highly multidrug resistant nature, S. aureus is a serious threat. It colonizes the nasal passages of approximately 30% of the healthy adult population. S. aureus infections initiate through trauma to the skin or mucosal layer and then progress through an invasive or toxin-mediated process. The prevalence of these infections has increased due to higher rates of immunosuppressive conditions, greater use of surgical implants, and dramatic increases in antibiotic resistance.

S. aureus produces an extensive array of enzymes, hemolysins, and toxins that are important to its ability to spread through tissues and cause disease. These virulence factors serve a wide scope of purposes in the infection process, including disruption of the epithelial barrier, inhibition of opsonization by antibody and complement, neutrophil cytolysis, interference with neutrophil chemotaxis, and inactivation of antimicrobial peptides. The expression of all of these invasive factors is controlled by cell-density quorum sensing using the autoinducing peptide (AIP) molecule. Like other quorum-sensing signals, AIP accumulates outside the cell until it reaches a critical concentration and then binds to a surface receptor called AgrC, initiating a regulatory cascade. Since AIP controls the expression of accessory factors for S. aureus, this regulatory system has been named the accessory gene regulator (agr), and the majority of the proteins necessary for this quorum-sensing system to function are encoded in the agr chromosomal locus. Applying inhibitors to quench this communication system to attenuate pathogenicity and virulence lies at the core of the quorum quenching approach.

Agr plays a key role in S. aureus pathogenesis. For example, skin and soft tissue infections are the most common type of infection caused by S. aureus. These range from minor inflammatory conditions to more invasive infection, and most of these cases are associated with the formation of abscesses, the hallmark of a S. aureus infection. The bulk of the phenotype is due to agr-dependent secreted virulence factors. Interference with the agr system through the use of competing AIPs or AIP-sequestering antibodies decreased abscess formation. These findings provide direct support for the notion that agr-targeted therapies could be an option for the development of skin infection treatments. Looking at other types of infections, agr mutants also display attenuated virulence in mice in the establishment of pneumonia and mortality, and in a systemic bloodstream infection model.

Given the importance of the agr system in pathogenesis, it has become the target of a number of chemical anti-virulence approaches. With the extracellular exposure of the AgrC receptor, chemists have developed receptor antagonists that successfully inhibit the system in vitro.

Castanea sativa leaves were identify as a potential source of anti-infective agents. Through design of a bioactivity-guided fractionation strategy based on limited growth-impact coupled to quorum sensing inhibition, a highly efficacious botanical composition with universal quenching activity was created for all agr alleles.

This inhibition of virulence and pathogenesis was accomplished without posing growth inhibitory pressures on not only S. aureus, but also a panel of common members of the human cutaneous microbiome. A robust skin microflora is critical to skin barrier health and prevention of disease onset. The majority of the bacterial cutaneous microbiome is represented by Actinobacteria, Firmicutes, Proteobacteria and Bacteroidetes. Much like cases of dysbiosis in gut microflora, broad-spectrum activity against the skin microflora also holds the potential for fostering an environment amenable to the proliferation of pathogenic bacteria. The presence of commensals, like Staphylococcus epidermidis, is essential to the state of host innate immunity. Thus, it is noteworthy that 224C-F2 specifically blocks S. aureus virulence without adding selective pressures on major representatives of the cutaneous microbiome.

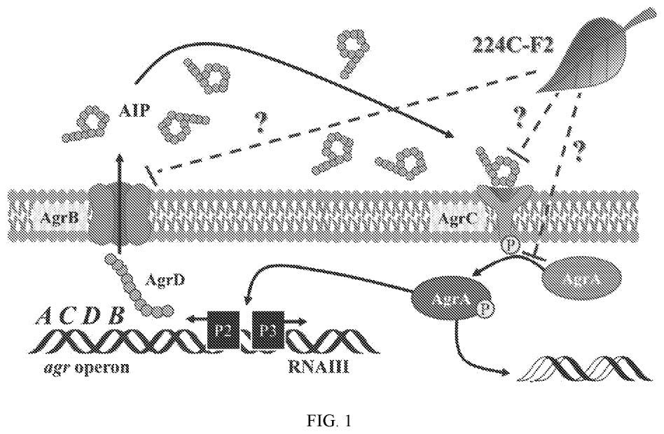

Multiple lines of evidence suggest that components within 224C-F2 directly target the core machinery of the agr system, such as the observation of agr P3 promoter reduction and reduced levels of δ-toxin production, which is encoded within RNAIII transcript regulated by P3. If 224C-F2 only targeted downstream factors regulated by quorum sensing, such as α-hemolysin, inhibition of agr P3 or δ-toxin production would not have been expected. Potential targets within the agr system include inhibition of AIP docking with AgrC, prevention of AIP production through AgrB, or reduction of AgrA activation (FIG. 1).

Following 15 days of sequential passaging with 224C-F2 in vitro, no resistance was detected. Thus, it is contemplated that 224C-F2 and compounds contained therein are a therapeutic option due to its ability to specifically target and quench S. aureus virulence. Importantly, this composition was non-toxic to human keratinocytes and no dermatopathology was noted upon administration to murine skin. Moreover, the composition did not inhibit growth of the normal skin microflora, suggesting that its disruptive action on the cutaneous microbiome would be minimal.

One major benefit of using virulence inhibitors with a classical antibiotic is the potential for increased antibiotic efficacy. By blocking the toxins responsible for immune response damage, the antibiotics and immune system can work more in concert to eliminate the bacteria.

Other topical formulations for skin flares (i.e. for atopic dermatitis or other infections related to a disrupted skin barrier) that may be combined with the anti-virulence drug include: topical steroids, anti-inflammatory agents, and promoters of skin barrier function or skin moisturizers such as ceramide, glycerin, colloidal oatmeal.

In certain embodiments the disclosure contemplates that an extract or one or more compounds in an extract disclosed herein may be used as a virulence inhibitor applications optionally in combination with other antibacterial agents for prevention of disease onset and treatment such as in medical device coatings (medical implants and tools, IV catheters), wound dressings (embedded in gauze bandages), wound rinses (i.e. surgical rinses), wound-vacuum systems, whole body baths (e.g., in combo with bleach baths for treatment of skin flares for atopic dermatitis/eczema), soaps, personal care products (body washes, lotions, soaps) for high risk patients or for populations with high risk of exposure (e.g. athletes using common sports equipment in gym) human and veterinary applications (e.g. anti-infectives for companion animals, race horses, etc.)

### Methods of Use

In certain embodiments, this disclosure relates to methods of treating or preventing bacterial infections comprising administering or contacting a formula comprising an extract or one or more compounds in an extract as disclosed herein to a subject in need thereof. In certain embodiments, the formula is administered in combination with another antibiotic agent.

In further embodiments, the subject is co-administered with an antibiotic selected from the group comprising of sulfonamides, diaminopyrimidines, quinolones, beta-lactam antibiotics, cephalosporins, tetracyclines, nitrobenzene derivatives, aminoglycosides, macrolide antibiotics, polypeptide antibiotics, nitrofuran derivatives, nitroimidazoles, nicotinic acid derivatives, polyene antibiotics, imidazole derivatives or glycopeptide, cyclic lipopeptides, glycylcyclines and oxazolidinones. In further embodiments, these antibiotics include but are not limited to sulfadiazine, sulfones-[dapsone (DDS) and para-aminosalicylic (PAS)], sulfanilamide, sulfamethizole, sulfamethoxazole, sulfapyridine, trimethoprim, pyrimethamine, nalidixic acids, norfloxacin, ciprofloxacin, cinoxacin, enoxacin, gatifloxacin, gemifloxacin, grepafloxacin, levofloxacin, lomefloxacin, moxifloxacin, ofloxacin, pefloxacin, sparfloxacin, trovafloxacin, penicillins (amoxicillin, ampicillin, azlocillin, carbenicillin, cloxacillin, dicloxacillin, flucloxacillin, hetacillin, oxacillin, mezlocillin, penicillin G, penicillin V, piperacillin), cephalosporins (cefacetrile, cefadroxil, cefalexin, cephaloglycin, cefalonium, cephaloridine, cefalotin, cefapirin, cefatrizine, cefazolin, cefazedone, cefazolin, cefradine, cefroxadine, ceftezole, cefaclor, cefonicid, ceforanide, cefprozil, cefuroxime, cefuzonam, cefmetazole, cefotetan, cefoxitin, cefcapene, cefdaloxime, cefdinir, cefditoren, cefetamet, cefixime, cefmenoxime, cefodizime, cefoperazone, cefotaxime, cefotiam, cefpiramide, cefpodoxime, cefteram, ceftibuten, ceftiofur, ceftaroline, ceftizoxime, ceftriaxone, cefoperazone, ceftazidime, cefepime), moxalactam, carbapenems (imipenem, ertapenem, meropenem) monobactams (aztreonam)oxytetracycline, chlortetracycline, clomocycline, demeclocycline, tetracycline, doxycycline, lymecycline, meclocycline, methacycline, minocycline, rolitetracycline, chloramphenicol, amikacin, gentamicin, framycetin, kanamycin, neomycin, netilmicin, streptomycin, tobramycin, azithromycin, clarithromycin, dirithromycin, erythromycin, roxithromycin, telithromycin, colistin, bacitracin, tyrothricin, nitrofurantoin, furazolidone, metronidazole, tinidazole, isoniazid, pyrazinamide, ethionamide, nystatin, amphotericin-B, hamycin, miconazole, clotrimazole, ketoconazole, fluconazole, rifampicin, lincomycin, clindamycin, spectinomycin, fosfomycin, loracarbef, polymyxin B, polymyxin B Sulfate, procaine, ramoplanin, teicoplanin, vancomycin, and/or nitrofurantoin.

In certain embodiments, this disclosure relates to methods of treating or preventing a toxin-mediated bacterial infection comprising administering an effective amount of a Castanea extract or compounds contained therein to a subject in need thereof, including a subject at risk of, exhibiting symptoms of, or diagnosed with a staphylococcal scalded skin syndrome (esp. in neonates), abscesses, necrotizing fasciitis, sepsis, atopic dermatitis (eczema) and more.

In certain embodiments, the subject is at risk of, exhibiting symptoms of, or diagnosed with toxic shock syndrome, scalded skin syndrome, abscesses, furuncles, cellulitis, folliculitis, bloodstream infections, medical device infections, pneumonia, osteomyelitis, staphylococcal food poisoning, skin and soft tissue infections, endocarditis, eczema, atopic dermatitis, psoriasis, impetigo, septic arthritis, brain abscess, burn wounds, venous ulcers, diabetic foot ulcers, surgical wounds, post-operation infections, carbuncles, meningitis, bacteremia, necrotizing pneumonia, or necrotizing fasciitis.

In certain embodiments, the disclosure contemplates methods of preventing bacterial infections by applying extracts or one or more compounds in extracts disclosed herein in a tampon for prevention against adverse effects associated with vaginal area infections and possibly bladder infections, e.g., toxic shock syndrome. As used herein a “tampon” refers to device containing an absorbent material, configured to be inserted into a vagina to absorb menstrual flow and typically expand during use, typically in the shape of a cylinder. Tampons may expand axially (increase in length), while digital tampons will expand radially (increase in diameter). Most tampons have a cord or string for removal. Typical tampon materials include cloth, fibers, cotton, or rayon, or a blend of rayon and cotton.

Bacterial toxins may cause toxic shock syndrome (TSS). Enterotoxin type B or TSST-1 of Staphylococcus aureus are believed to cause TSS. Streptococcal TSS is sometimes referred to as toxic shock-like syndrome (TSLS) or streptococcal toxic shock syndrome (STSS). CDC criteria for diagnosing staphylococcal toxic shock syndrome is based on 1) a body temperature of greater than 38.9° C. (102.02° F.) 2) a Systolic blood pressure of greater than 90 mmHg 3) diffuse macular erythroderma 4) desquamation (especially of the palms and soles) 1-2 weeks after onset 5) involvement of three or more organ systems: gastrointestinal (vomiting, diarrhea), muscular: severe myalgia or creatine phosphokinase level at least twice the upper limit of normal for laboratory, mucous membrane hyperemia (vaginal, oral, conjunctival), kidney failure (serum creatinine>2 times normal), liver inflammation (bilirubin, AST, or ALT>2 times normal), low platelet count (platelet count<100,000/mm³), Central nervous system involvement (confusion without any focal neurological findings) and 6) Negative results of: blood, throat, and CSF cultures for other bacteria (besides S. aureus) negative serology for Rickettsia infection, leptospirosis, and measles. Cases are classified as probable is five of the six criteria above are met.

In certain embodiments, the disclosure contemplates methods of preventing general transmission of bacterial through use of extracts and one or more compounds in an extract disclosed herein as a general agent formulated into a spray or wipe product, paper or fiber-based cloth. For example, one can use such a product to treat athletic equipment (football pads, bench presses, gym surfaces), where invasive toxin mediated staph often lurks and causes infections in healthy people through toxin production.

In certain embodiments, the disclosure relates to methods of treating acne comprising administering an effective amount of a composition comprising an extract or one or more compounds in an extract as disclosed herein to a subject at risk of, exhibiting symptoms of, or diagnosed with acne, blackheads, papules, pustules or nodules. In certain embodiments, the subject is undergoing puberty, between 10 and 20 years of age. In certain embodiments, the subject is a female, and the composition is administered within seven days of the beginning of a menstrual cycle. Administration may be by topical application through hand or by spray of a liquid or lotion containing an extract or one or more compounds in an extract disclosed herein.

### Extracts and Compounds

In certain embodiments, an extract is made by the process of extracting a mixture of compounds from the leaves, roots, bark, stem, or branches of a Castanea plant e.g., Castanea sativa. Other contemplated plants include: Castanea acuminatissima, Castanea alabamensis, Castanea alnifolia, Castanea americana, Castanea argentea, Castanea argyrophylla, Castanea arkansana, Castanea armata, Castanea ashei, Castanea blaringhemii, Castanea bodinieri, Castanea brevicuspis, Castanea bungeana, Castanea burbankii, Castanea buruana, Castanea californica, Castanea Castanea, Castanea castanicarpa, Castanea castenea var. pubinervis, Castanea chincapin, Castanea chinensis, Castanea chrysophylla, Castanea concinna, Castanea cooperta, Castanea costata, Castanea coudersii, Castanea crenata, Castanea davidii, Castanea dentata, Castanea diversifolia, Castanea dovaricata, Castanea duclouxii, Castanea echidnocarpa, Castanea edonii, Castanea edwii, Castanea endicottii, Castanea eonii, Castanea fagus, Castanea falconeri, Castanea fargesii, Castanea fauriei, Castanea fleetii, Castanea floridana, Castanea formosana, Castanea furfurella, Castanea glomerata, Castanea henryi, Castanea henryi, Castanea hupehensis, Castanea hystrix, Castanea, Castanea inermis, Castanea japonica, Castanea javanica, Castanea kusakuri, Castanea lanceifolia, Castanea latifolia, Castanea margaretta, Castanea martabanica, Castanea microcarpa, Castanea mollissima, Castanea montana, Castanea morrisii, Castanea nana, Castanea neglecta, Castanea ozarkensis, Castanea paucispina, Castanea phansipanensis, Castanea prolifera, Castanea pubinervis, Castanea pulchella, Castanea pumila, Castanea purpurella, Castanea regia, Castanea rhamnifolia, Castanea rockii, Castanea roxburghii, Castanea seguinii, Castanea sempervirens, Castanea sessilifolia, Castanea sinensis, Castanea sloanea, Castanea spectabilis, Castanea sphaeroarpa, Castanea sphaerocarpa, Castanea stricta, Castanea sumatrana, Castanea tribuloides, Castanea tungurrut, Castanea vesca, Castanea vilmoriniana, Castanea vulgaris, Castanea wattii. and hybrids thereof.

In certain embodiments, the extracting process comprises the step of mixing the leaf from the plant with a polar solvent, such as a liquid comprising methanol, ethanol, ethyl acetate, acetonitrile, acetone, methylene chloride or chloroform, under conditions such that a mixture of compounds in the leaf dissolves in the solvent. In certain embodiments, the process further comprises the step of removing the solvent by evaporation from the mixture of compounds. In certain embodiments, the process further comprises the step of purifying the mixture of compounds by liquid chromatography through a solid absorbent, e.g., wherein the solid absorbent comprises silica gel or alumina.

In certain embodiments, the disclosure relates to extracts comprising a leaf derived mixture of compounds from a Castanea plant wherein the extracting process comprises the steps of: mixing a leaf with methanol under conditions such that leaf compounds dissolves in the methanol and removing the methanol providing a methanol derived mixture of compounds; partitioning the methanol derived mixture of compounds in hexane and water providing a water derived mixture of compounds; partitioning the water derived mixture of compounds by mixing the water with ethyl acetate under conditions such that leaf compounds dissolve in the ethyl acetate and removing the ethyl acetate providing an ethyl acetate derived mixture of compounds; and purifying the ethyl acetate derived mixture of compounds by liquid chromatography through silica with a mobile phase comprising hexane and ethylene acetate; wherein the mobile phase comprises increasing amounts of ethyl acetate, and a mobile phase fraction is isolated comprising a leaf derived mixture of compounds which does not contain chlorogenic acid, ellagic acid, hyperoside, isoquercitrin, or rutin.

Chromatography refers to the separation of a mixture of compounds dissolved in a fluid called the mobile phase, which carries the compounds through a structure holding another material called the stationary phase. The various compounds or components of the mixture travel at different speeds, causing them to separate. The separation is based on differential partitioning between the mobile and stationary phases. Subtle differences in a partition coefficient of each compound result in differential retention on the stationary phase and thus changing the separation.

In normal-phase chromatography, the stationary phase is polar. In reversed phase, the stationary phase is nonpolar. Typical stationary phases for normal-phase chromatography are silica or organic moieties with cyano and amino functional groups. For reversed phase, alkyl hydrocarbons are the preferred stationary phase. Examples are solid supports containing a surface conjugated with a hydrocarbon chain, e.g., octadecyl (C18), octyl (C8), and butyl (C4).

In normal-phase chromatography, the least polar compounds elute first, and the most polar compounds elute last. The mobile phase typically consists of a nonpolar solvent such as hexane or heptane mixed with a slightly more polar solvent such as isopropanol, ethyl acetate or chloroform. Retention to the stationary phase decreases as the amount of polar solvent in the mobile phase increases. In reversed phase chromatography, the most polar compounds elute first with the most nonpolar compounds eluting last. The mobile phase is generally a binary mixture of water and a miscible polar organic solvent like methanol, acetonitrile or THF.

In certain embodiments, methods of extraction comprise mixing leaves of a Castanea plant with an water miscible carbon containing solvent, e.g., such as a protic solvent, an alcohol, methanol, ethanol, 1-propanol, 2-propanol, tetrahydrofuran, acetone, acetic acid, 1,4-dioxane or mixture providing a concentrate with a mixture of compounds and substantially removing the solvent from the concentrate, purifying the solvent derived concentrate to less than 5%, 1%, or 0.5% by weight of the solvent used in the extraction, e.g., evaporating the protic solvent and/or optionally in combination with mixing the concentrate with water, sonicating the water, freezing the water to provide ice, and removing the ice by sublimation (e.g. in a vacuum of low pressure) wherein said purification methods may be repeated in combination. In certain embodiments, the method further comprises suspending the solvent derived concentrate in water and optionally extract impurities in a hydrocarbon solvent such as cyclohexane, heptane, hexane, pentane, 2,2,4-trimethylpentane, separating the hydrocarbon from the water providing a water layer. In certain embodiments, the method further comprises mixing the water layer with a solvent that is immiscible in water (polar and/or aprotic), e.g., such as ethyl acetate, diethyl ether, methyl tertbutyl ether, toluene, methylene chloride, carbon tetrachloride, 1,2-dichloroethant, and/or chloroform, and purifying the solvent to provide a second solvent derived concentrate. In further embodiments, the second derived concentrate is purified one or more times by liquid chromatography, e.g., normal phase chromatography. Typically, the solid absorbent is polar such as silica. In certain embodiments, the extract is a portion isolated after the column solvent is more than 50% ethyl acetate in hexane.

### Pharmaceutical Formulation

In certain embodiments, the disclosure relates to a pharmaceutical formulation comprising an extract or one or more compounds in an extract disclosed herein and a pharmaceutically acceptable excipient or additive. In certain embodiments, the disclosure relates to a lotion, liquid, or gel formulation optionally further comprising an antibiotic agent, a topical steroid, an anti-inflammatory agent, a promoter of skin barrier function, a skin moisturizer or combinations thereof.

Examples of antibiotics include but are not limited to sulfadiazine, sulfones-[dapsone (DDS) and para aminosalicylic (PAS)], sulfanilamide, sulfamethizole, sulfamethoxazole, sulfapyridine, trimethoprim, pyrimethamine, nalidixic acids, norfloxacin, ciprofloxacin, cinoxacin, enoxacin, gatifloxacin, gemifloxacin, grepafloxacin, levofloxacin, lomefloxacin, moxifloxacin, ofloxacin, pefloxacin, sparfloxacin, trovafloxacin, penicillins (amoxicillin, ampicillin, azlocillin, carbenicillin, cloxacillin, dicloxacillin, flucloxacillin, hetacillin, oxacillin, mezlocillin, penicillin G, penicillin V, piperacillin), cephalosporins (cefacetrile, cefadroxil, cefalexin, cephaloglycin, cefalonium, cefaloridine, cefalotin, cefapirin, cefatrizine, cefazolin, cefazedone, cefazolin, cefradine, cefroxadine, ceftezole, cefaclor, cefonicid, ceforanide, cefprozil, cefuroxime, cefuzonam, cefmetazole, cefotetan, cefoxitin, cefcapene, cefdaloxime, cefdinir, cefditoren, cefetamet, cefixime, cefmenoxime, cefodizime, cefoperazone, cefotaxime, cefotiam, cefpiramide, cefpodoxime, cefteram, ceftibuten, ceftiofur, ceftizoxime, ceftriaxone, cefoperazone, ceftazidime, cefepime), moxalactam, carbapenems (imipenem, ertapenem, meropenem) monobactams (aztreonam)oxytetracycline, chlortetracycline, clomocycline, demeclocycline, tetracycline, doxycycline, lymecycline, meclocycline, methacycline, minocycline, rolitetracycline, chloramphenicol, amikacin, gentamicin, framycetin, kanamycin, neomycin, netilmicin, streptomycin, tobramycin, azithromycin, clarithromycin, dirithromycin, erythromycin, roxithromycin, telithromycin, colistin, bacitracin, tyrothricin, nitrofurantoin, furazolidone, metronidazole, tinidazole, isoniazid, pyrazinamide, ethionamide, nystatin, amphotericin-B, hamycin, miconazole, clotrimazole, ketoconazole, fluconazole, rifampicin, lincomycin, clindamycin, spectinomycin, fosfomycin, loracarbef, polymyxin B, polymyxin B Sulfate, procaine, ramoplanin, teicoplanin, vancomycin, and/or nitrofurantoin.

Examples of steroids include hydrocortisone, hydrocortisone valerate, hydrocortisone 17-butyrate, mometasone, mometasone furoate, halobetasol propionate, desonide, desoximetasone, fluocinolone acetonide, alclometasone dipropionate, flurandrenolide, fluticasone propionate, diflucortolone, diflucortolone valerate, diflorasone diacetate, clobetasol, clobetasone butyrate, clobetasol propionate, betamethasone dipropionate, betamethasone valerate, beclomethasone, budesonide, flunisolide, fluocinonide, triamcinolone, triamcinolone acetonide, methylprednisolone, methylprednisolone aceponate, prednicarbate, prednisolone, and prednisone and alternate salts thereof. Examples of contemplated anti-inflammatory agents are aspirin, celecoxib, diclofenac, diflunisal, etodolac, ibuprofen, indomethacin, ketoprofen, naproxen, oxaprozin, and piroxicam.

In certain embodiments, the disclosure relates to a pharmaceutical composition comprising an extract or one or more compounds in an extract disclosed herein formulated with an enteric coating. In certain embodiments, the disclosure relates to a pharmaceutical formulation of an extract or one or more compounds in an extract disclosed herein which protect the compositions from the acidity and enzymatic action of gastric secretions. In certain embodiments, the pharmaceutical formulations contain an extract or one or more compounds in an extract disclosed herein in a composition with an enteric coating along with another pharmaceutically acceptable vehicle. In certain embodiments, compositions comprising an extract or one or more compounds in an extract disclosed herein may be directly compressible without excipients, into a tablet of pharmaceutically acceptable hardness, e.g., compressed into a tablet, optionally with a lubricant, such as but not limited to magnesium stearate, and enteric coated. In another embodiment, the pharmaceutical compositions containing an extract or one or more compounds in an extract disclosed herein alternatively include one or more substances that either neutralize stomach acid and/or enzymes or are active to prevent secretion of stomach acid.

The pharmaceutical composition can be formulated for oral administration as, for example but not limited to, drug powders, crystals, granules, small particles (which include particles sized on the order of micrometers, such as microspheres and microcapsules), particles (which include particles sized on the order of millimeters), beads, microbeads, pellets, pills, microtablets, compressed tablets or tablet triturates, molded tablets or tablet triturates, and in capsules, which are either hard or soft and contain the composition as a powder, particle, bead, solution or suspension. The pharmaceutical composition can also be formulated for oral administration as a solution or suspension in an aqueous liquid, as a liquid incorporated into a gel capsule or as any other convenient formulation for administration, or for rectal administration, as a suppository, enema or other convenient form.

The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

Suitably, the pharmaceutical composition of the disclosure comprises a carrier and/or diluent appropriate for administration by injection to a human or animal organism. Such carrier and/or diluent is non-toxic at the dosage and concentration employed. It is selected from those usually employed to formulate compositions for parental administration in either unit dosage or multi-dose form or for direct infusion by continuous or periodic infusion. It is typically isotonic, hypotonic or weakly hypertonic and has a relatively low ionic strength, such as provided by sugars, polyalcohols and isotonic saline solutions. Representative examples include sterile water, physiological saline (e.g. sodium chloride), bacteriostatic water, Ringer's solution, glucose or saccharose solutions, Hank's solution, and other aqueous physiologically balanced salt solutions. The pH of the composition of the disclosure is suitably adjusted and buffered in order to be appropriate for use in humans or animals, typically at a physiological or slightly basic pH (between about pH 8 to about pH 9, with a special preference for pH 8.5). Suitable buffers include phosphate buffer (e.g. PBS), bicarbonate buffer and/or Tris buffer. A typical composition is formulated in 1M saccharose, 150 mM NaCl, 1 mM MgCl₂, 54 mg/l Tween 80, 10 mM Tris pH 8.5. Another typical composition is formulated in 10 mg/ml mannitol, 1 mg/ml HSA, 20 mM Tris, pH 7.2, and 150 mM NaCl.

The pharmaceutical formulation can also include any type of pharmaceutically acceptable excipients, additives or vehicles. For example, but not by way of limitation, diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropyl methylcellulose may be added to the composition comprising an extract or one or more compounds in an extract disclosed herein to increase the bulk of the composition. Also, binders, such as but not limited to, starch, gelatin, sucrose, glucose, dextrose, molasses, lactose, acacia gum, sodium alginate, extract of Irish moss, carboxymethylcellulose, methylcellulose, polyvinylpyrrolidone, polyethylene glycol, ethylcellulose, and waxes, may be added to the formulation to increase its cohesive qualities. Additionally, lubricants, such as but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate, and magnesium lauryl sulfate may be added to the formulation. Also, glidants, such as but not limited to, colloidal silicon dioxide or talc may be added to improve the flow characteristics of a powdered formulation. Finally, disintegrants, such as but not limited to, starches, clays, celluloses, algins, gums, crosslinked polymers (e.g., croscarmellose, crospovidone, and sodium starch glycolate), Veegum, methylcellulose, agar, bentonite, cellulose and wood products, natural sponge, cation-exchange resins, alginic acid, guar gum, citrus pulp, carboxymethylcellulose, or sodium lauryl sulfate with starch may also be added to facilitate disintegration of the formulation in the intestine.

In certain embodiments, the formulation contains a directly compressible composition comprising an extract or one or more compounds in an extract disclosed herein but no excipients, additives or vehicles other than an enteric coating; however, the formulation may contain a lubricant, such as but not limited to, magnesium stearate. Preferably, the directly compressed formulation is formulated as a tablet of pharmaceutically acceptable hardness (greater than 6 kp, preferably 8-14 kp, and more preferably 10-13 kp).

Polymers which are useful for the preparation of enteric coatings include, but are not limited to, shellac, starch and amylose acetate phthalates, styrene-maleic acid copolymers, cellulose acetate succinate, cellulose acetate phthalate (CAP), polyvinyl acetate phthalate (PVAP), hydroxypropyl methylcellulose phthalate (grades HP-50 and HP-55), ethylcellulose, fats, butyl stearate, and methacrylic acid-methacrylic acid ester copolymers with acid ionizable groups (“EUDRAGIT™”), such as “EUDRAGIT™ L 30D”, “EUDRAGIT™ RL 30D”, “EUDRAGIT™ RS 30D”, “EUDRAGIT™ L 100-55”, and “EUDRAGIT™ L 30D-55”.

Application of the enteric coating to composition can be accomplished by any method known in the art for applying enteric coatings. For example, but not by way of limitation, the enteric polymers can be applied using organic solvent-based solutions containing from 5 to 10% w/w polymer for spray applications and up to 30% w/w polymer for pan coatings. Solvents that are commonly in use include, but are not limited to, acetone, acetone/ethyl acetate mixtures, methylene chloride/methanol mixtures, and tertiary mixtures containing these solvents. Some enteric polymers, such as methacrylic acid-methacrylic acid ester copolymers can be applied using water as a dispersant.

Furthermore, plasticizers can be added to the enteric coating to prevent cracking of the coating film. Suitable plasticizers include the low molecular weight phthalate esters, such as diethyl phthalate, acetylated monoglycerides, triethyl citrate, polyethyl glycol tributyl citrate and triacetin. Generally, plasticizers are added at a concentration of 10% by weight of enteric coating polymer weight. Other additives such as emulsifiers, for example detergents and simethicone, and powders, for example talc, may be added to the coating to improve the strength and smoothness of the coating. Additionally, pigments may be added to the coating to add color to the pharmaceutical formulation.

In certain embodiments, the composition comprising an extract or one or more compounds in an extract disclosed herein is formulated with a compound or compounds which neutralize stomach acid. Alternatively, the pharmaceutical composition containing an extract or one or more compounds in an extract disclosed herein is administered either concurrent with or subsequent to administration of a pharmaceutical composition which neutralize stomach acid. Compounds, such as antacids, which are useful for neutralizing stomach acid include, but are not limited to, aluminum carbonate, aluminum hydroxide, bismuth subnitrate, bismuth subsalicylate, calcium carbonate, dihydroxyaluminum sodium carbonate, magaldrate, magnesium carbonate, magnesium hydroxide, magnesium oxide, and mixtures thereof.

In certain embodiments, composition comprising an extract or one or more compounds in an extract disclosed herein is administered with a substance that inactivates or inhibits the action of stomach enzymes, such as pepsin. Alternatively, the pharmaceutical composition containing the proanthocyanidin polymer composition is administered either concurrent with or subsequent to administration of a pharmaceutical composition active to inactivate or inhibit the action of stomach enzymes. For example, but not by way of limitation, protease inhibitors, such as aprotinin, can be used to inactivate stomach enzymes.

In certain embodiments, the composition comprising an extract or one or more compounds in an extract disclosed herein is formulated with a compound or compounds which inhibit the secretion of stomach acid. Alternatively, the pharmaceutical composition is administered either concurrent with or subsequent to administration of a pharmaceutical composition active to inhibit the secretion of stomach acid. Compounds which are useful for inhibiting the secretion of stomach acid include, but are not limited to, ranitidine, nizatidine, famotidine, cimetidine, and misoprostol.

### Cosmetic Formulations and Personal Care Products

In certain embodiments, the disclosure relates to a cosmetic formulation comprising an extract or one or more compounds in an extract disclosed herein and cosmetically acceptable excipient or additive. In certain embodiments, the disclosure relates to a solid or liquid soap or lotion comprising an extract or one or more compounds in an extract disclosed herein and a fatty acid.

In certain embodiments, additives can be selected from the group consisting of oily bodies, surfactants, emulsifiers, fats, waxes, pearlescent waxes, bodying agents, thickeners, superfatting agents, stabilizers, polymers, silicone compounds, lecithins, phospholipids, biogenic active ingredients, deodorants, antimicrobial agents, antiperspirants, film formers, antidandruff agents, swelling agents, insect repellents, hydrotropes, solubilizers, preservatives, perfume oils and dyes.

In certain embodiments, additives are selected from the group consisting of surfactants, emulsifiers, fats, waxes, stabilizers, deodorants, antiperspirants, antidandruff agents and perfume oils.

As used herein, cosmetic preparations can mean care agents. Care agents are understood as meaning care agents for skin and hair. These care agents include, inter alia, cleansing and restorative action for skin and hair.

In certain embodiments, preparations may be cosmetic and/or dermopharmaceutical preparations, e. g. hair shampoos, hair lotions, foam baths, shower baths, creams, gels, lotions, alcoholic and aqueous/alcoholic solutions, emulsions, wax/fat compositions, stick preparations, powders or ointments.

Surfactants (or Surface-active substances) that may be present are anionic, non-ionic, cationic and/or amphoteric surfactants, the content of which in the compositions is usually about 1 to 70% by weight, preferably 5 to 50% by weight and in particular 10 to 30% by weight. Typical examples of anionic surfactants are soaps, alkylbenzenesulfonates, alkanesulfonates, olefin sulfonates, alkyl ether sulfonates, glycerol ether sulfonates, α-methyl ester sulfonates, sulfo fatty acids, alkyl sulphates, fatty alcohol ether sulphates, glycerol ether sulphates, fatty acid ether sulphates, hydroxy mixed ether sulphates, monoglyceride (ether) sulphates, fatty acid amide (ether) sulphates, mono- and dialkyl sulfosuccinates, mono- and dialkyl sulfosuccinamates, triglycerides, amide soaps, ether carboxylic acids and salts thereof, fatty acid isethionates, fatty acid sarcosinates, fatty acid taurides, N-acylamino acids, e.g. acyl lactylates, acyl tartrates, acyl glutamates and acyl aspartates, alkyl oligoglucoside sulphates, protein fatty acid condensates (in particular wheat-based vegetable products) and alkyl (ether) phosphates. If the anionic surfactants contain polyglycol ether chains, these may have a conventional homologous distribution, but preferably have a narrowed homologous distribution. Typical examples of non-ionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucuronic acid derivatives, fatty acid N-alkyl glucamides, protein hydrolysates (in particular wheat-based vegetable products), polyol fatty acid esters, sugar esters, sorbitan esters, polysorbates and amine oxides. If the non-ionic surfactants contain polyglycol ether chains, these may have a conventional homologous distribution, but preferably have a narrowed homologous distribution. Typical examples of cationic surfactants are quaternary ammonium compounds, e.g. dimethyl distearyl ammonium chloride, and ester-quats, in particular quaternized fatty acid trialkanolamine ester salts. Typical examples of amphoteric or zwitterionic surfactants are alkyl betaines, alkyl amido betaines, amino propionates, amino glycinates, imidazolinium-betaines and sulfobetaines. Said surfactants are known compounds. With regard to structure and preparation of these substances, reference may be made to relevant review works.

Typical examples of particularly suitable mild, i.e. particularly skin-compatible surfactants are fatty alcohol polyglycol ether sulphates, monoglyceride sulphates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkyl amido betaines, amphoacetates and/or protein fatty acid condensates, the latter preferably based on wheat proteins.

Suitable oily bodies are, for example, alcohols based on fatty alcohols having 6 to 18, preferably 8 to 10, carbon atoms, esters of linear C6-C22-fatty acids with linear or branched C6-C22-fatty alcohols or esters of branched C6-C13-carboxylic acids with linear or branched C6-C22-fatty alcohols, for example myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C6-C22-fatty acids with branched alcohols, in particular 2-ethylhexanol, esters of C18-C38-alkylhydroxycarboxylic acids with linear or branched C6-C22-fatty alcohols, in particular dioctyl maleates, esters of linear and/or branched fatty acids with polyhydric alcohols (for example propylene glycol, dimer diol or trimer triol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, for example dicaprylyl carbonates (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or unsymmetrical dialkyl ethers having 6 to 22 carbon atoms per alkyl group, for example dicaprylyl ether (Cetiol® OE), ring-opening products of epoxidized fatty acid esters with polyols, silicone oils (cyclomethicones, silicon methicone types, inter alia) and/or aliphatic or naphthenic hydrocarbons, for example squalene or dialkyl cyclohexanes.

Suitable emulsifiers are, for example, non ionogenic surfactants from at least one of the following groups:

addition products of from 2 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group, and onto alkylamines having 8 to 22 carbon atoms in the alkyl radical;

alkyl and/or alkenyl oligoglycosides having 8 to 22 carbon atoms in the alk(en)yl radical and the ethoxylated analogs thereof;

addition products of from 1 to 15 mol of ethylene oxide onto castor oil and/or hydrogenated castor oil;

addition products of from 15 to 60 mol of ethylene oxide onto castor oil and/or hydrogenated castor oil;

partial esters of glycerol and/or sorbitan with unsaturated, linear or saturated, branched fatty acids having 12 to 22 carbon atoms and/or hydroxycarboxylic acids having 3 to 18 carbon atoms, and the adducts thereof with 1 to 30 mol of ethylene oxide;

partial esters of polyglycerol (average degree of self-condensation 2 to 8), polyethylene glycol (molecular weight 400 to 5000), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and the adducts thereof with 1 to 30 mol of ethylene oxide;

mixed esters of pentaerythritol, fatty acids, citric acid and fatty alcohols and/or mixed esters of fatty acids having 6 to 22 carbon atoms, methylglucose and polyols, preferably glycerol or polyglycerol,

mono-, di- and trialkyl phosphates, and mono-, di- and/or tri-PEG alkyl phosphates and salts thereof;

wool wax alcohols;

polysiloxane-polyalkyl-polyether copolymers and corresponding derivatives;

block copolymers, e.g. polyethylene glycol-30 dipolyhydroxystearates;

polymer emulsifiers, e.g. Pemulen® grades (TR-1, TR-2) from Goodrich;

polyalkylene glycols and glycerol carbonate.

The addition products of ethylene oxide and/or of propylene oxide onto fatty alcohols, fatty acids, alkylphenols or onto castor oil are known, commercially available products. These are homologous mixtures whose average degree of alkoxylation corresponds to the ratio of the amounts of ethylene oxide and/or propylene oxide and substrate with which the addition reaction is carried out. C12/18-fatty acid mono- and diesters of addition products of ethylene oxide onto glycerol are known as refatting agents for cosmetic preparations.

Alkyl and/or alkenyl oligoglycosides can be prepared by reacting glucose or oligosaccharides with primary alcohols having 8 to 18 carbon atoms. With regard to the glycoside radical, both monoglycosides, in which a cyclic sugar radical is glycosidically bonded to the fatty alcohol, and also oligomeric glycosides having a degree of oligomerization of up to, preferably, about 8, are suitable. The degree of oligomerization here is a statistical average value that is based on a homologous distribution customary for such technical-grade products.

Typical examples of suitable partial glycerides are hydroxy stearic acid monoglyceride, hydroxy stearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linoleic acid monoglyceride, linoleic acid diglyceride, erucic acid monoglyceride, erucic acid diglyceride, tartaric acid monoglyceride, tartaric acid diglyceride, citric acid monoglyceride, citric acid diglyceride, malic acid mono-glyceride, malic acid diglyceride, and the technical-grade mixtures thereof which may also comprise small amounts of triglyceride as a minor product of the preparation process. Likewise, suitable are addition products of 1 to 30 mol, preferably 5 to 10 mol, of ethylene oxide onto said partial glycerides.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and technical-grade mixtures thereof. Likewise, suitable are addition products of 1 to 30 mol, preferably 5 to 10 mol, of ethylene oxide onto said sorbitan esters.

Typical examples of suitable polyglycerol esters are polyglyceryl-2 dipolyhydroxystearate (Dehymuls® PGPH), polyglycerol-3 diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (Polyglycerol Caprate T2010/90), polyglyceryl-3 cetyl ether (Chimexane® NL), polyglyceryl-3 distearate (Cremophor® GS 32) and polyglyceryl polyricinoleate (Admul® WOL 1403), polyglyceryl dimerate isostearate, and mixtures thereof. Examples of further suitable polyol esters are the mono-, di- and triesters, optionally reacted with 1 to 30 mol of ethylene oxide, of trimethylolpropane or pentaerythritol with lauric acid, coconut fatty acid, tallow fatty acid, palmitic acid, stearic acid, oleic acid, behenic acid and the like.

Furthermore, zwitterionic surfactants can be used as emulsifiers. The term “zwitterionic surfactants” refers to those surface-active compounds that carry at least one quaternary ammonium group and at least one carboxylate and one sulfonate group in the molecule. Particularly suitable zwitterionic surfactants are the betaines, such as N-alkyl-N,N-dimethylammonium glycinates, for example coco alkyl dimethyl ammonium glycinate, N-acylaminopropyl-N,N-dimethylammonium glycinates, for example cocoacyl amino propyldimethylammonium glycinate, and 2-alkyl-3-carboxymethyl-3-hydroxyethylimidazolines having in each case 8 to 18 carbon atoms in the alkyl or acyl group, and coco acylamino ethylhydroxyethyl carboxymethyl glycinate. Particular preference is given to the fatty acid amide derivative known under the CTFA name Cocamidopropyl Betaine. Likewise, suitable emulsifiers are ampholytic surfactants. The term “ampholytic surfactants” means those surface-active compounds that, apart from a C_{8/18}-alkyl or -acyl group in the molecule, contain at least one free amino group and at least one —CO₂H or SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyl-taurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkyl aminopropionate, cocoacyl-aminoethyl aminopropionate and C_{12/18}-acylsarcosine. Finally, cationic surfactants are also suitable emulsifiers, those of the ester quat type, preferably methyl-quaternized difatty acid triethanolamine ester salts, being particularly preferred.

Fats and waxes that can be used are described in the following text. Typical examples of fats are glycerides, i.e. solid or liquid vegetable or animal products which consist essentially of mixed glycerol esters of higher fatty acids, suitable waxes are inter alia natural waxes, for example candelilla wax, carnauba wax, japan wax, esparto grass wax, cork wax, rice germ oil wax, sugarcane wax, beeswax, shellac wax, spermaceti, lanolin (wool wax), uropygial grease, ceresin, ozokerite (earth wax), petrolatum, paraffin waxes, microcrystalline waxes; chemically modified waxes (hard waxes), for example montan ester waxes, sasol waxes, hydrogenated jojoba waxes, and synthetic waxes, for example polyalkylene waxes and polyethylene glycol waxes. In addition to the fats, suitable additives are also fat-like substances, such as lecithins and phospholipids.

The term “lecithins” is understood by the person skilled in the art as meaning those glycerophospholipids which form from fatty acids, glycerol, phosphoric acid and choline by esterification. Lecithins are thus frequently also known as phosphatidylcholines (PC). Examples of natural lecithins which may be mentioned are the cephalins, which are also referred to as phosphatidic acids and represent derivatives of 1,2-diacyl-sn-glycerol-3-phosphoric acids. By contrast, phospholipids are usually understood as meaning mono- and, preferably, diesters of phosphoric acid with glycerol (glycerophosphates), which are generally considered to be fats. In addition, sphingosines and sphingolipids are also suitable.

Examples of suitable pearlescent waxes are: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, for example fatty alcohols, fatty ketones, fatty aldehydes, fatty ethers and fatty carbonates, which have a total of at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Bodying agents and thickeners that can be used are described in the following text. Suitable bodying agents are primarily fatty alcohols or hydroxy fatty alcohols having 12 to 22, and preferably 16 to 18, carbon atoms, and also partial glycerides, fatty acids or hydroxy fatty acids. Preference is given to a combination of these substances with alkyl oligoglucosides and/or fatty acid N-methylglucamides of identical chain length and/or polyglycerol poly-12-hydroxystearates. Suitable thickeners are, for example, hydrophilic silicas, polysaccharides, in particular xanthan gum, guar, agar, alginates and tyloses, carboxymethylcellulose and hydroxyethylcellulose, and also relatively high molecular weight polyethylene glycol mono- and diesters of fatty acids, polyacrylates, polyacrylamides, polymers, polyvinyl alcohol and polyvinylpyrrolidone, surfactants, for example ethoxylated fatty acid glycerides, esters of fatty acids with polyols for example pentaerythritol or trimethylolpropane, fatty alcohol ethoxylates having a narrowed homolog distribution or alkyl oligoglucosides, and electrolytes such as sodium chloride and ammonium chloride.

Superfatting agents which can be used are for example lanolin and lecithin, and polyethoxylated or acylated lanolin and lecithin derivatives, polyol fatty acid esters, monoglycerides and fatty acid alkanolamides, the latter also serving as foam stabilizers.

Stabilizers which can be used are metal salts of fatty acids, for example magnesium, aluminium and/or zinc stearate or ricinoleate.

Polymers that can be used are described in the following text. Suitable cationic polymers are, for example, cationic cellulose derivatives, for example a quaternized hydroxyethylcellulose, copolymers of diallylammonium salts and acryl amides, quaternized vinylpyrrolidone-vinylimidazole polymers, for example Luviquat® (BASF), condensation products of polyglycols and amines, quaternized collagen polypeptides, for example lauryldimonium hydroxypropyl hydrolysed collagen (Lamequat® L/Grunau), quaternized wheat polypeptides, polyethyleneimine, cationic silicone polymers, for example amodimethicones, copolymers of adipic acid and dimethylaminohydroxypropyldiethylenetriamine (Cartaretins®/Sandoz), copolymers of acrylic acid with dimethyl diallylammonium chloride (Merquat® 550/Chemviron), polyaminopolyamides and cross linked water-soluble polymers thereof, cationic chitin derivatives, for example quaternized chitosan, optionally in microcrystalline dispersion, condensation products from dihaloalkyls, for example dibromobutane with bisdialkylamines, for example bis-dimethylamino-1,3-propane, cationic guar gum, for example Jaguar® CBS, Jaguar® C-17, Jaguar® C-16 from Celanese, quaternized ammonium salt polymers.

Suitable anionic, zwitterionic, amphoteric and nonionic polymers are, for example, vinyl acetate-crotonic acid copolymers, vinylpyrrolidone-vinyl acrylate copolymers, vinyl acetate-butyl maleate-isobornyl acrylate copolymers, methyl vinyl ether-maleic anhydride copolymers and esters thereof, uncrosslinked polyacrylic acids and polyacrylic acids crosslinked with polyols, acrylamidopropyltrimethylammonium chloride-acrylate copolymers, octylacrylamide-methyl methacrylate-tert-butylamino-ethyl methacrylate-2-hydroxypropyl methacrylate copolymers, polyvinylpyrrolidone, vinylpyrrolidone-vinyl acetate copolymers, vinylpyrrolidone-dimethylaminoethyl methacrylate-vinylcaprolactam terpolymers, and optionally derivatized cellulose ethers and silicones.

Suitable silicone compounds are, for example, dimethylpolysiloxanes, methylphenylpolysiloxanes, cyclic silicones, and amino-, fatty-acid-, alcohol-, polyether-, epoxy-, fluorine-, glycoside- and/or alkyl-modified silicone compounds, which can either be liquid or in resin form at room temperature. Also suitable are simethicones, which are mixtures of dimethicones having an average chain length of from 200 to 300 dimethylsiloxane units and hydrogenated silicates.

Deodorants and antimicrobial agents that can be used are described in the following text. Cosmetic deodorants counteract, mask or remove body odors. Body odors arise as a result of the effect of skin bacteria on apocrine perspiration, with the formation of degradation products which have an unpleasant odor. Accordingly, deodorants comprise active ingredients which act as antimicrobial agents, enzyme inhibitors, odor absorbers or odor masking agents. Suitable antimicrobial agents are, in principle, all substances effective against gram-positive bacteria, for example 4-hydroxybenzoic acid and its salts and esters, N-(4-chlorophenyl)-N′-(3,4-dichloro-phenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methyl-ethyl)phenol, 2-benzyl-4-chlorophenol, 3-(4-chlorophenoxy)-1,2-propanediol, 3-iodo-2-propynyl butylcarbamate, chlorohexidine, 3,4,4′-trichlorocarbanilide (TTC), antibacterial fragrances, thymol, thyme oil, eugenol, oil of cloves, menthol, mint oil, farnesol, phenoxyethanol, glycerol monocaprate, glycerol monocaprylate, glycerol monolaurate (GML), diglycerol monocaprate (DMC), salicylic acid N-alkylamides, for example n-octylsalicylamide or n-decylsalicylamide.

Suitable enzyme inhibitors are, for example, esterase inhibitors. These are preferably trialkyl citrates, such as trimethyl citrate, tripropyl citrate, triisopropyl citrate, tributyl citrate and, in particular, triethyl citrate (Hydagen® CAT). The substances inhibit enzyme activity, thereby reducing the formation of odor. Other substances which are suitable esterase inhibitors are sterol sulfates or phosphates, for example lanosterol, cholesterol, campesterol, stigmasterol and sitosterol sulfate or phosphate, dicarboxylic acids and esters thereof, for example glutaric acid, monoethyl glutarate, diethyl glutarate, adipic acid, monoethyl adipate, diethyl adipate, malonic acid and diethyl malonate, hydroxycarboxylic acids and esters thereof, for example citric acid, malic acid, tartaric acid or diethyl tartrate, and zinc glycinate.

Suitable odor absorbers are substances which are able to absorb and largely retain odor-forming compounds. They lower the partial pressure of the individual components, thus also reducing their rate of diffusion. It is important that in this process perfumes must remain unimpaired. Odor absorbers are not effective against bacteria. They comprise, for example, as main constituent, a complex zinc salt of ricinoleic acid or specific, largely odor-neutral fragrances which are known to the person skilled in the art as “fixatives”, for example extracts of labdanum or styrax or certain abietic acid derivatives. The odor masking agents are fragrances or perfume oils, which, in addition to their function as odor masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal raw materials, for example civet and castoreum. Typical synthetic fragrance compounds are products of the ester, ether, aldehyde, ketone, alcohol and hydro-carbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styralyl propionate and benzyl salicylate. The ethers include, for example, benzyl ethyl ether, and the aldehydes include, for example, the linear alkanals having 8 to 18 carbon atoms, citral, citronellal, citronellyl oxyacetaldehyde, cyclamen aldehyde, hydroxycitronellal, lilial and bourgeonal, the ketones include, for example, the ionones and methyl cedryl ketone, the alcohols include anethole, citronellol, eugenol, isoeugenol, geraniol, linalool, phenylethyl alcohol and terpineol, and the hydrocarbons include mainly the terpenes and balsams. Preference is, however, given to using mixtures of different fragrances which together produce a pleasing fragrance note. Ethereal oils of relatively low volatility, which are mostly used as aroma components, are also suitable as perfume oils, e.g. sage oil, chamomile oil, oil of cloves, melissa oil, mint oil, cinnamon leaf oil, linden flower oil, juniper berry oil, vetiver oil, olibanum oil, galbanum oil, labdanum oil and lavandin oil. Preference is given to using bergamot oil, dihydromyrcenol, lilial, citronellol, phenylethyl alcohol, α-hexylcinnamaldehyde, geraniol, benzylacetone, cyclamen aldehyde, linalool, boisambrene forte, ambroxan, indole, sandelice, lemon oil, mandarin oil, orange oil, allyl amyl glycolate, cyclovertal, lavandin oil, clary sage oil, β-damascone, geranium oil bourbon, cyclohexyl salicylate, iraldein gamma, phenylacetic acid, geranyl acetate, benzyl acetate, rose oxide, irotyl and floramat alone or in mixtures.

Antiperspirants reduce the formation of perspiration by influencing the activity of the eccrine sweat glands, thus counteracting underarm wetness and body odor. Aqueous or anhydrous formulations of antiperspirants typically comprise one or more of the following ingredients: astringent active ingredients, oil components, nonionic emulsifiers, co-emulsifiers, bodying agents, auxiliaries, for example thickeners or complexing agents, and/or nonaqueous solvents, for example ethanol, propylene glycol and/or glycerol.

Suitable astringent antiperspirant active ingredients are primarily salts of aluminum, zirconium or of zinc. Such suitable antihydrotic active ingredients are, for example, aluminum chloride, aluminum chlorohydrate, aluminum dichlorohydrate, aluminum sesquichlorohydrate and complex compounds thereof, e.g. with 1,2-propylene glycol, aluminum hydroxy allantoinate, aluminum chloride tartrate, aluminum zirconium trichlorohydrate, aluminum zirconium tetrachlorohydrate, aluminum zirconium pentachlorohydrate and complex compounds thereof, e.g. with amino acids, such as glycine. In addition, customary oil-soluble and water-soluble auxiliaries may be present in antiperspirants in relatively small amounts. Such oil-soluble auxiliaries may, for example, be anti-inflammatory, skin-protective or perfumed ethereal oils, synthetic skin-protective active ingredients and/or oil-soluble perfume oils.

Customary water-soluble additives are, for example, preservatives, water-soluble fragrances, pH regulators, e.g. buffer mixtures, water-soluble thickeners, e.g. water-soluble natural or synthetic polymers, for example xanthan gum, hydroxyethylcellulose, polyvinylpyrrolidone or high molecular weight polyethylene oxides.

Film formers that can be used are described in the following text. Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid and salts thereof, and similar compounds.

Suitable antidandruff active ingredients are piroctone olamine (1-hydroxy-4-methyl-6-(2,4,4-trimethylpentyl)-2-(1H)-pyridinone monoethanolamine salt), Baypival® (climbazole), Ketoconazole (4-acetyl-1-{-4-[2-(2,4-dichlorophenyl) r-2-(1H-imidazol-1-ylmethyl]-1,3-dioxylan-c-4-ylmethoxyphenyl}piperazine, ketoconazole, elubiol, selenium disulfide, colloidal sulfur, sulfur polyethylene glycol sorbitan monooleate, sulfur ricinol polyethoxylate, sulfur tar distillates, salicyclic acid (or in combination with hexachlorophene), undecylenic acid monoethanolamide sulfosuccinate Na salt, Lamepon® UD (protein undecylenic acid condensate), zinc pyrithione, aluminum pyrithione and magnesium pyrithione/dipyrithione magnesium sulfate.

Suitable insect repellents are N,N-diethyl-m-toluamide, 1,2-pentanediol or ethyl butylacetylaminopropionate.

To improve the flow behavior, hydrotropes, for example ethanol, isopropyl alcohol, or polyols, can be used. Polyols which are suitable here preferably have 2 to 15 carbon atoms and at least two hydroxyl groups. The polyols can also contain further functional groups, in particular amino groups, or be modified with nitrogen. Typical examples are: glycerol; alkylene glycols, for example, ethylene glycol, diethylene glycol, propylene glycol, butylene glycol, hexylene glycol, and polyethylene glycols with an average molecular weight of from 100 to 1 000 daltons; technical-grade oligoglycerol mixtures with a degree of self-condensation of from 1.5 to 10, for example, technical-grade diglycerol mixtures with a diglycerol content of from 40 to 50% by weight; methylol compounds, such as trimethylolethane, trimethylolpropane, trimethylol-butane, pentaerythritol and dipentaerythritol; lower alkyl glucosides, in particular those with 1 to 8 carbon atoms in the alkyl radical, for example methyl and butyl glucoside; sugar alcohols with 5 to 12 carbon atoms, for example sorbitol or mannitol, sugars with 5 to 12 carbon atoms, for example glucose or sucrose; amino sugars, for example glucamine; dialcohol amines, such as diethanolamine or 2-amino-1,3-propanediol.

### Medical Device Coatings, Wound Dressings, and Irrigation

In certain embodiments, the disclosure relates to a medical device comprising a coating comprising an extract or one or more compounds in an extract disclosed herein optionally in combination with another antibiotic. In certain embodiments, the medical device is an ear tube, eye lenses, contact lenses, coronary stent, metal screw, pin, plate, rod, catheter, artificial knee, cardioverter defibrillator, artificial hip, heart pacemaker, breast implant, spine screws, rods, and discs, intra-uterine devices

In certain embodiments, the disclosure relates to a wound dressing comprising an extract or one or more compounds in an extract disclosed herein wherein the wound dress comprises an absorbent pad and optionally an adhesive optionally in combination with another antibiotic agent. In certain embodiments, the wound dressing is a foam or compression dressing or a cover dressing such as wraps, gauze and tape.

In certain embodiments, the wound dressing comprises alginate or collagen.

In certain embodiments, the wound dressing a hydrocolloid dressing, e.g., carboxymethylcellulose and gelatin optionally in a polyurethane foam or film, optionally comprising one or more agents selected from, pectin, polysaccharides, and an adhesive.

In certain embodiments, the wound dressing is a hydrogel. Hydrogels are polymers that contain a high content, e.g., greater than 40, 50, 60, 70, 80, 90, or 95%, of hydroxy and/or carboxyl containing monomers or salts thereof, e.g., vinyl alcohol, acrylic acid, 2-hydroxyethylmethacrylate monomers, which can be co-polymers to provide varying degrees of hydration, e.g., copolymerization with ethylene glycol dimethacrylate. Due to the hydrophilic monomers, the hydrogels typically absorb water to contain greater than 70, 80, 85, 90, 95% water by weight. Contemplated hydrogel dressings include: amorphous hydrogel, which are a free-flowing gel that are typically distributed in tubes, foil packets and spray bottles; an impregnated hydrogel, which are typically saturated onto a gauze pad, nonwoven sponge ropes and/or strips; or a sheet hydrogel which are gel held together by a fiber mesh.

A flow of wound rinse/irrigation solution is applied across an open wound surface to achieve wound hydration, to remove deeper debris, and to assist with the visual examination. In certain embodiments, the disclosure relates to methods of irrigating using a solution comprising an extract or one or more compound in an extract disclosed herein. In certain embodiments, the disclosure relates to a wound rinse comprising an extract or one or more compounds in an extract disclosed herein optionally in combination with normal saline, sterile water, detergent, surfactant, preservatives, or iodine.

In certain embodiments, the disclosure contemplates a kit comprising a container comprising an extract or one or more compounds in an extract discloses herein optionally comprising a second container comprising a solution, normal saline, sterile water, detergent, surfactant, preservatives, iodine, hydrogen peroxide, or sodium hypochlorite.

### Examples

### Hydroperoxy-Cycloartanes

Hydroperoxy-cycloartanes were isolated from the methanolic extract of the leaves of Castanea sativa, a substantial, long-lived deciduous tree that produces an edible seed, the chestnut. 224C-F2c-PF42-SF6 is a mixture of a cycloartane with a hydroperoxy group at the terminus of its alkyl chain and a carboxylic acid and methyl group replacing the gem-dimethyl group (1a) and a cycloartane with a terminal alkene and carboxylic acid in the S configuration at C24 (1b). 224C-F2c-PF42-SF7 is a cycloartane with a terminal alkene and carboxylic acid in the R configuration at C24 (2). The structure and absolute configuration of 224C-F2c-PF42-SF7 (2) was elucidated via X-ray crystallography, NMR spectroscopy, and MS; while that of 224C-F2c-PF42-SF6 (1a and 1b) was elucidated via X-ray crystallography (1a), 1D and 2D NMR spectroscopic experiments and FTMS. Due to the photooxidation presented in FIG. 5 compounds 1a and 1b could not be separated chromatographically and were tested as the mixture SF6. The compounds 2 and SF6 were evaluated for the ability to inhibit fluorescence of Staphylococcus aureus reporter strains of agr, the main gene regulating quorum sensing. Additionally, 2 and SF6 were evaluated for cytotoxicity against human immortalized keratinocytes (HaCaT). Both moderately inhibit fluorescence across agr reporter strains with an IC50 of 8 μg/mL, a concentration at which they exert no growth inhibitory effects on S. aureus.

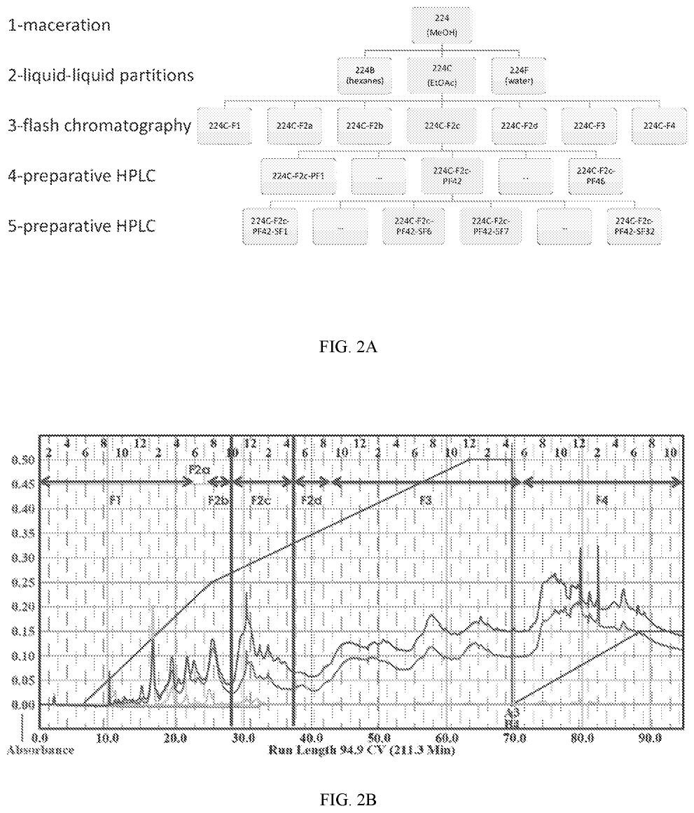

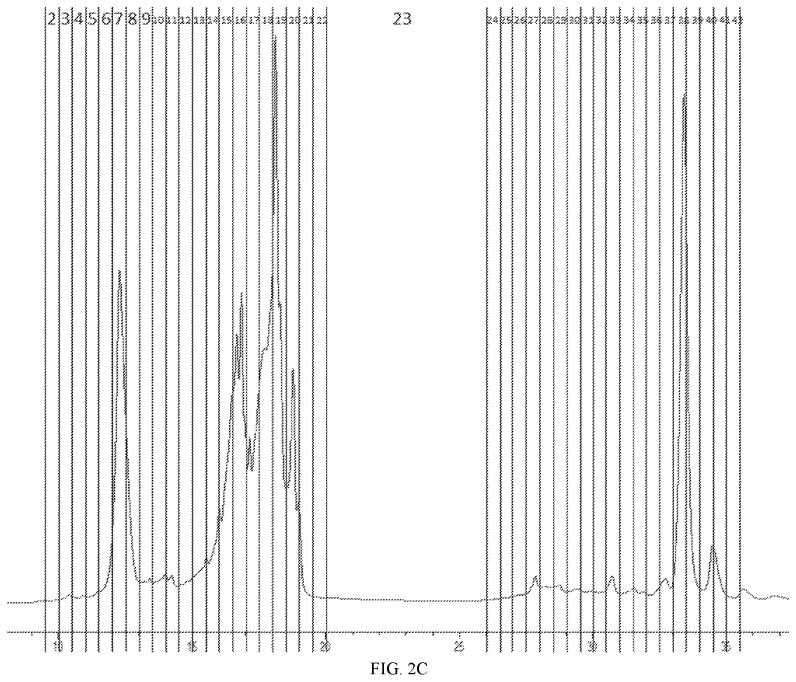

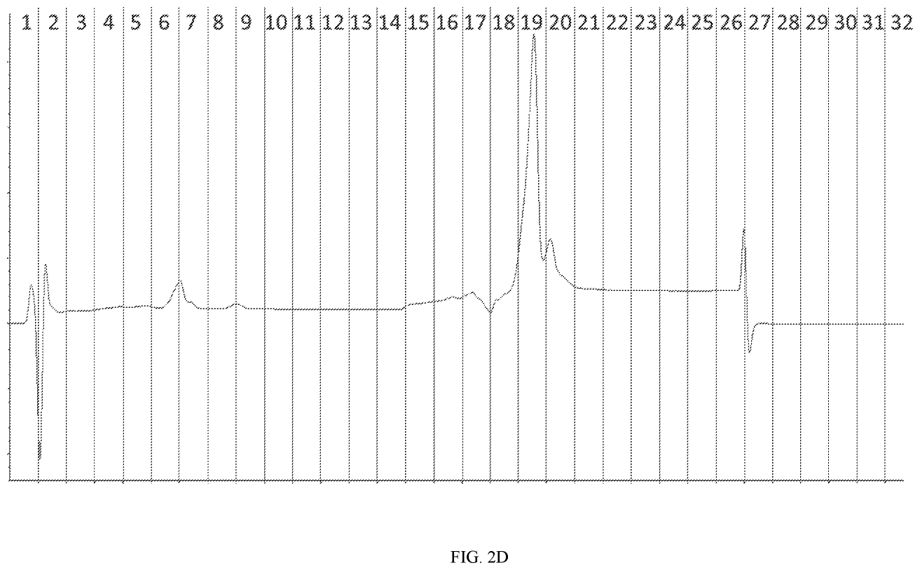

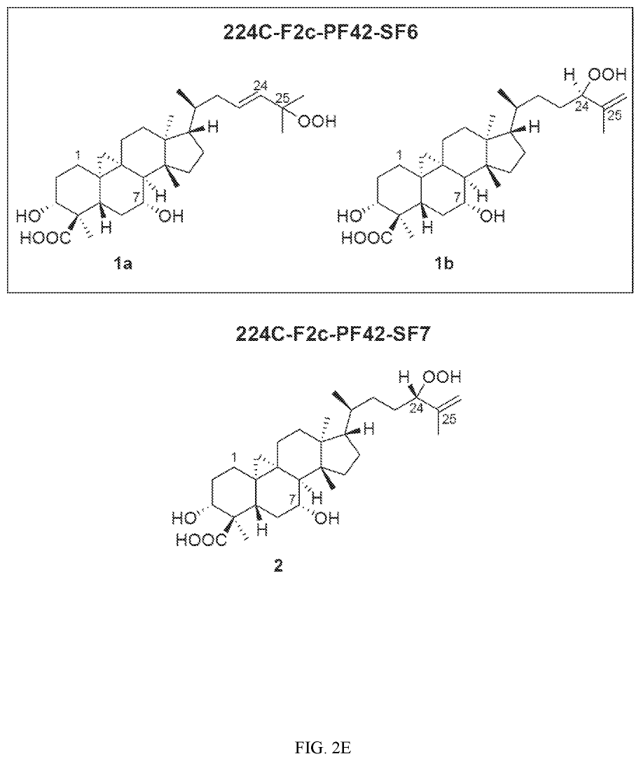

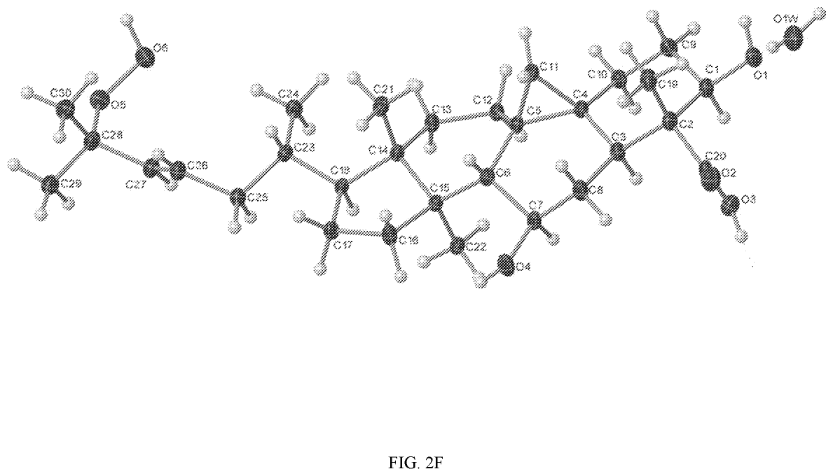

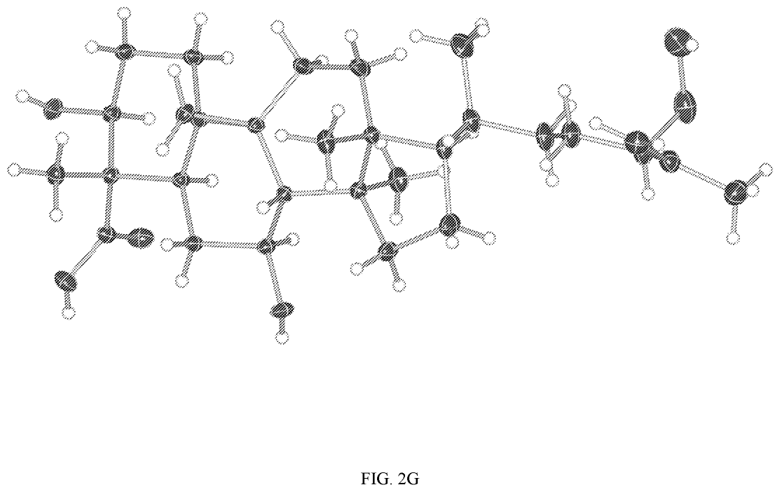

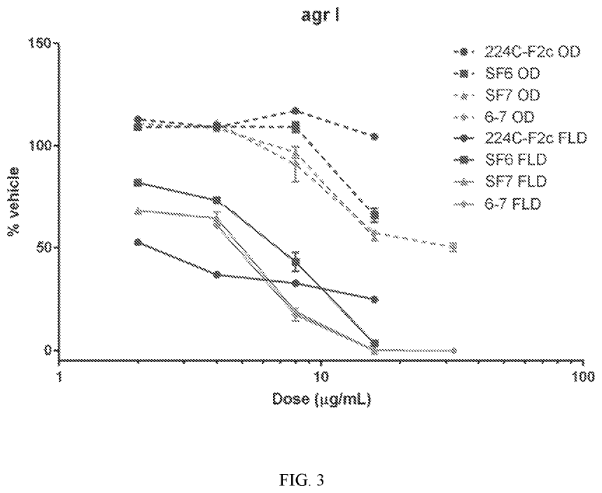

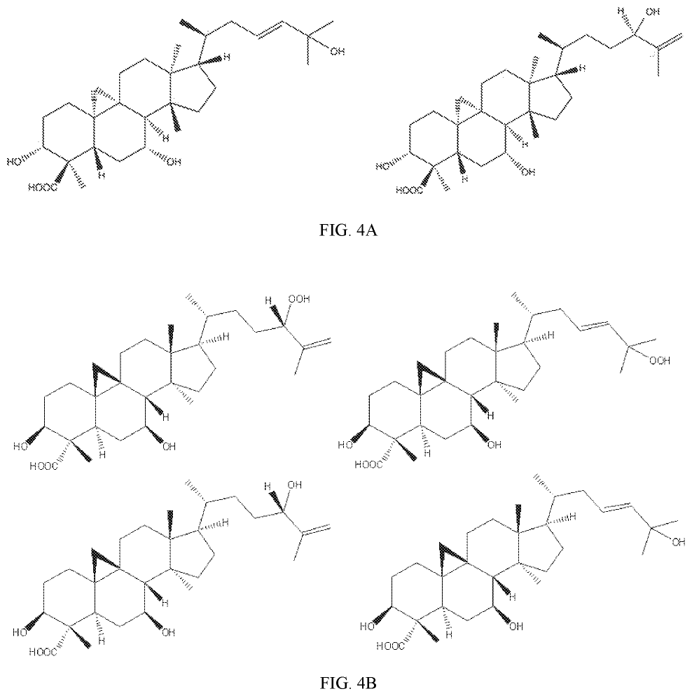

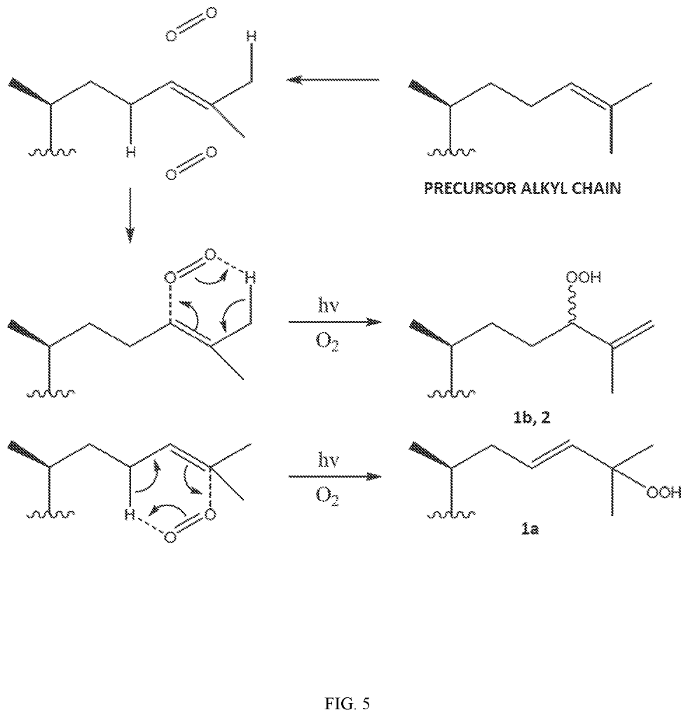

After eluting from the column, 224C-F2 was split into parts a-d. The partition 224C was split into 7 fractions by flash chromatography (-F1, -F2a, -F2b, -F2c, -F2d, -F3, and -F4). A screen against S. aureus reporter strains of AIP-induced agr:P3 activation representing all four agr subtypes functioned as a preliminary readout of quorum sensing inhibition and the bioassay which guided fractionation. 224C-F2c was found to be the most active fraction of 224C. It was subsequently fractionated via reverse phase HPLC to produce fractions, called preparative fractions (PFs) (FIG. 2C). After screening the 43 PFs in the aforementioned reporter strain assay, PF22-28, 39-42 were identified as most bioactive. 224C-F2c-PF42 (abbreviated as PF42), was fractionated via a second round of reverse phase HPLC into sub-fractions (SFs) (FIG. 2D). From 224C-F2c-PF42, 224C-F2c-PF42-SF6 (1a and 1b, abbreviated as SF6) and 224C-F2c-PF42-SF7 (2, abbreviated as SF7) were isolated.

### Chromatography

Ground, dried leaves of C. sativa were macerated in MeOH at room temperature for two successive periods of 72 h, with daily agitation. Filtered extracts were concentrated in vacuo, lyophilized, then partitioned in succession with hexanes and ethyl acetate. The resulting nonaqueous partitions were dried over anhydrous Na2SO4, concentrated in vacuo, and lyophilized before testing for activity. The ethyl acetate partition (224C) was subjected to further fractionation using a CombiFlash™ Rf+ (Teledyne ISCO) flash chromatography system using a RediSep™ Rf Gold silica column. Extract 224C was bonded to Celite 545 (Acros Organics) at a 1:4 ratio and dry-loaded using a RediSep™ dry load cartridge. The mobile phase consisted of (A) hexane, (B) EtOAc, and (C) MeOH. The linear gradient begins with 100% A for 6.3 column volumes (CV), and then increased to 50:50 A:B by25.3 CV, and increased to 100% B at 63.3 CV, which was held until 69.6 CV, and then to 70:30 B:C at 88.6 CV, which was held until 94.9 CV. The chromatography was monitored at 254 and 280 nm. 224C-F2 represents the eluate obtained from 23.0 min to 43.0 min.

224C-F2c represents the eluate obtained from the chromatography when 28.0 column volumes to 37.0 column volumes of mobile phase had eluted. Method development for fractionation via HPLC was performed on an Agilent 1260 Infinity system running OpenLab™ CDS ChemStation™ (Agilent Technologies, Santa Clara, Calif., USA) with an Agilent Eclipse™ XDB-C18 (250 mm×4.6 mm, 5 μm) column with compatible guard column at a column temperature of 25° C. Mobile phase reagents were HPLC-grade and purchased from Fisher Scientific, except for the Type 1 water, which was obtained from an EMD Millipore MILLI-Q™ water system (Billerica, Mass.). Mobile phase consisted of a linear gradient elution 0.1% formic acid in water (A) and 0.1% formic acid in acetonitrile (B) at a flow rate of 1 mL/min. Production of PFs of 224C-F2c was performed on an Agilent 1260 Infinity II system running the same software. The column used was an Agilent XDB-C18 (250 mm×30 mm, 5 μm) column. Initial conditions were 98:2 (A:B), these were held for 5.55 min, then changed to 43:57 (A:B) using a linear gradient at 14.55 min, which was held until 23.55 min, then changed to 100% B at 26.55 min by a linear gradient, the final conditions were held until 45 min. Samples were prepared in MeOH and 2 mL injections were made. Chromatograms were monitored at 254 nm and 314 nm. A second round of HPLC fractionation, to split the PFs into SFs, utilized an Agilent Prep-C18 (50 mm×30 mm, 5 μm) column. Initial conditions were 70:30 (A:B) changing to 43:57 (A:B) at 2 min and held until 6.5 min, then changed to 100% B by linear gradient at 8 min and held until 12.5 min, before returning to initial conditions. Samples were prepared in MeOH and 1 mL injections were made. Chromatograms were monitored at 217 nm and 254 nm.

High resolution mass spectrometry was performed using a Thermo Scientific LTQ-FT Ultra™ MS equipped with a nanospray source in negative mode and processed with Thermo Scientific Xcalibur™ 2.2 SP1.48 software (San Jose, Calif.). The liquid sample was placed into a New Objective Econo Picotip™ (Fisher Scientific) which was inserted into the nanosource using a static nanospray probe. Nitrogen was used as a backing pressure to provide flow to the tip.

### Compound 2

Compound 2 was obtained as a colorless oil from the 224C-F2c-PF42-SF7 fraction. The HRMS-APCI for compound 2 was m/z 503.3381 [M-H]- (calcd for C₃₀H₄₇O₆, 503.3373). The 1D NMR data of compound 2 (Table 1) resembled those of musambin A, except for the change in the position of a hydroxyl group, at C-7, instead of C-1 in musambin A. The structure and stereochemistry of compound 2 was confirmed by COSY, HSQC, HMBC and NOESY experiments and X-ray crystallography. The structures of musambin A and B are reported in Lacroix et al. Hydroperoxy-cycloartane triterpenoids from the leaves of Markhamia lutea, a plant ingested by wild chimpanzees, Phytochemistry, 70 (2009) 1239-1245.

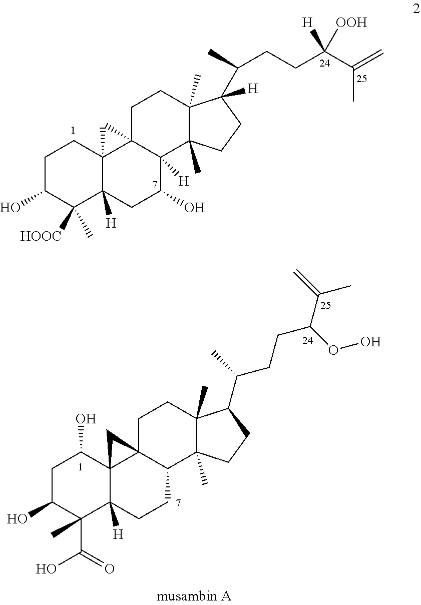

TABLE 1
¹H NMR and ¹³C NMR data for compound 2 (600
MHz) and Musambin A (400 MHz) in CD₃OD.

| Musambin A | | | | | | |
|---|---|---|---|---|---|---|
| Posi- | | | | Compound 2 | | |
| tion | δ_{H}, mult (J in Hz) | | δ_{C} | δ_{H}, mult (J in Hz) | | δ_{C} |
| 1 | 3.54 dd (J = 3.2; 2.8 Hz) | | 73.6 | 1.65 | m | 31.9 |
| | | | | 1.37 | m | |
| 2 | 1.84 ddd (J = 13.5; 4.9; | | 37.8 | 1.73 | m | 30.2 |
| | 3.2 Hz) | | | 1.60 | m | |
| | 1.79 ddd (J = 13.5; 12.0; | | | | | |
| | 2.8 Hz) | | | | | |
| 3 | 4.53 dd (J = 12.0; | | 71.4 | 4.00 d (J = 11.6 Hz, 1H) | | 76.4 |
| | 4.9 Hz) | | | | | |
| 4 | | | 55.9 | — | | 55.6 |
| 5 | 2.58 dd (J = 12.5; | | 38.3 | 2.13 d (J = 12.7 Hz, 1H) | | 43.3 |
| | 4.2 Hz) | | | | | |
| 6 | 1.30 | m | 24.0 | 1.45 | m | 33.3 |
| | 1.00 | m | | 1.11 | m | |
| 7 | 1.30 | m | 26.5 | 3.54 | m | 70.7 |
| | 1.30 | m | | | | |
| 8 | 1.50 | m | 50.1 | 1.73 | m | 54.5 |
| 9 | | | 22.2 | — | | 29.2 |
| 10 | | | 30.2 | — | | 21.3 |
| 11 | 2.40 ddd (J = 15.0; 9.0; | | 26.7 | 1.78 | m | 27.6 |
| | 9.0 Hz) | | | 1.44 | m | |
| | 1.30 | m | | | | |
| 12 | 1.70 dd (J = 9.0; | | 34.1 | 1.60 | m | 33.9 |
| | 9.0 Hz) | | | | | |
| 13 | | | 46.4 | — | | 46.7 |
| 14 | | | 50.0 | — | | 49.9 |
| 15 | 1.30 | m | 36.9 | 1.59 | m | 36.9 |
| | | | | 1.47 | m | |
| 16 | 1.91 | m | 29.1 | 1.65 | m | 28.1 |
| | 1.34 | m | | | | |
| 17 | 1.60 | m | 53.6 | 1.56 | m | 52.8 |
| 18 | 1.01 | s | 18.8 | 1.00 | s | 17.2 |
| 19 | 0.70 d (J = 4.5 Hz) | | 31.1 | 0.80 (d, J = 4.6 Hz, 1H) | | 27.1 |
| | 0.50 d (J = 4.5 Hz) | | | 0.30 (d, J = 4.6 Hz, 1H) | | |
| 20 | 1.45 | m | 37.1 | 1.41 | m | 37.4 |
| 21 | 0.90 d (J = 6.4 Hz) | | 18.7 | 0.90 (d, J = 6.4 Hz, 3H) | | 19.0 |
| 22 | 1.50 | m | 33.2 | 1.60 | m | 33.7 |
| | 1.00 | m | | | | |
| 23 | 1.50 | m | 28.3 | 1.89 | m | 28.7 |
| | 1.50 | m | | 1.31 | m | |
| 24 | 4.17 dd (J = 6.6; | | 91.0 | 4.16 t (J = 6.8 Hz, 1H) | | 90.9 |
| | 6.6 Hz) | | | | | |
| 25 | | | 145.7 | — | | 146.1 |
| 26 | 4.94 dq (J = 1.5; | | 114.3 | 4.91 | m | 114.0 |
| | 1.5 Hz) | | | 4.93 | m | |
| | 4.91 | m | | | | |
| 27 | 1.71 dd (J = 1.5; | | 16.9 | 1.71 | s | 17.1 |
| | 1.5 Hz) | | | | | |
| 28 | | | 180.6 | — | | 182.6 |
| 29 | 1.07 | s | 9.1 | 1.08 | s | 10.3 |
| 30 | 0.99 | s | 19.9 | 0.95 | s | 19.2 |

Considering that the molecular formula involves six oxygen atoms and that the structure encloses only three oxymethine groups in addition to a carboxyl group, it is suggested that CH-24 is linked to a hydroperoxide group. This is confirmed by the chemical shift of the carbon C-24 at δ_{C} 90.8 ppm, which is not that of a secondary alcohol function expected at δ_{C} 76.8 ppm, but rather that of a carbon bearing an hydroperoxide group which is expected at δ_{C} 90.4 ppm.

Compounds 1a and 1b

Compounds 1a and 1b from the 224C-F2c-PF42-SF6 fraction were isolated as a colorless amorphous solid. The HRMS-APCI for 1a and 1b was m/z 503.3398 [M-H]- (calcd for C30H47O6, 503.3373). The 1D NMR spectral data (Table 2) of compound 1a resemble those of the known compound musambin B, except for the change in the position of a hydroxyl group, at C-7 in 1a, instead of C-1 in musambin B. The structure of compound 1a was confirmed by COSY, HSQC and HMBC experiments. Also, the structure and stereochemistry of compound 1a was verified by X-ray experiments.

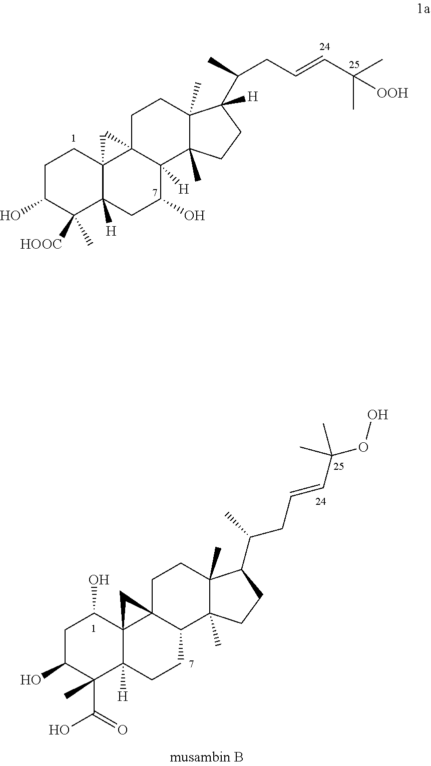

TABLE 2
¹H NMR and ¹³C NMR data for compound 1a (600
MHz) and musambin B (400 MHz) in CD₃OD.

| Musambin B | | | | | | |
|---|---|---|---|---|---|---|
| Posi- | | | | Compound 1a | | |
| tion | δ_{H}, mult (J in Hz) | | δ_{C} | δ_{H}, mult (J in Hz) | | δ_{C} |
| 1 | 3.54 dd (J = 3.0; | | 73.6 | 1.69 | m | 31.9 |
| | 3.0 Hz) | | | 1.40 | m | |
| 2 | 1.82 | m | 37.8 | 1.61 | m | 30.2 |
| | 1.77 | m | | 1.74 | m | |
| 3 | 4.53 dd (J = 11.9; | | 71.4 | 4.01 (dd, J = 11.6, | | 76.2 |
| | 4.9 Hz) | | | 4.0 Hz, 1H) | | |
| 4 | | | 55.9 | — | | 55.3 |
| 5 | 2.57 dd (J = 12.5; | | 38.3 | 2.14 (dd, J = 13.2, | | 43.4 |
| | 4.2 Hz) | | | 3.7 Hz, 1H) | | |
| 6 | 1.30 | m | 24.0 | 1.42 | m | 33.3 |
| | 1.00 | m | | 1.14 | m | |
| 7 | 1.30 | m | 26.5 | 3.53 | m | 70.5 |
| | 1.30 | m | | | | |
| 8 | 1.52 dd (J = 11.8; | | 50.1 | 1.74 | m | 54.5 |
| | 4.5 Hz) | | | | | |
| 9 | | | 22.2 | — | | 21.4 |
| 10 | | | 30.3 | — | | 27.5 |
| 11 | 2.42 ddd (J = 15.0; 8.5; | | 26.7 | 1.78 | m | 28.1 |
| | 8.5 Hz) | | | 1.49 | m | |
| | 1.25 m | | | | | |
| 12 | 1.69 dd (J = 8.5; | | 34.0 | 1.60 | m | 33.6 |
| | 8.5 Hz) | | | | | |
| 13 | | | 46.4 | — | | 46.7 |
| 14 | | | 50.0 | — | | 49.8 |
| 15 | 1.35 | m | 36.9 | 1.42 | m | 37.0 |
| 16 | 1.95 | m | 29.0 | 1.95 | m | 29.1 |
| | 1.35 | m | | 1.33 | m | |
| 17 | 1.61 ddd (J = 6.5; 6.5; | | 53.3 | 1.58 | m | 52.5 |
| | 6.5 Hz) | | | | | |
| 18 | 1.01 | s | 18.9 | 1.02 | s | 17.3 |
| 19 | 0.70 d (J = 4.5 Hz) | | 31.1 | 0.80 (d, J = 4.7 Hz, 1H) | | 269 |
| | 0.50 d (J = 4.5 Hz) | | | 0.30 (d, J = 4.6 Hz, 1H) | | |
| 20 | 1.45 | m | 37.6 | 1.51 | m | 37.7 |
| 21 | 0.92 d (J = 6.4 Hz) | | 18.9 | 0.90 (d, J = 6.8 Hz, 3H) | | 19.0 |
| 22 | 2.21 | m | 40.7 | 2.22 | m | 40.5 |
| | 1.80 | m | | 1.82 | m | |
| 23 | 5.63 ddd (J = 15.9; 7.0; | | 129.7 | 5.58 | m | 137.1 |
| | 5.0 Hz) | | | | | |
| 24 | 5.56 d (J = 15.9 Hz) | | 137.0 | 5.60 | m | 129.7 |
| 25 | | | 82.5 | — | | 82.5 |
| 26 | 1.29 | s | 24.9 | 1.29 | s | 24.9 |
| 27 | 1.29 | s | 24.9 | 1.29 | s | 25.1 |
| 28 | | | 180.8 | — | | 180.9 |
| 29 | 1.07 | s | 9.0 | 1.10 | s | 9.8 |
| 30 | 0.99 | s | 19.8 | 0.94 | s | 19.2 |

The assignment of the hydroperoxide group at C-25 was deduced in agreement with the observed chemical shift value of this carbon at 82.5 ppm, which is characteristic of a quaternary δ_{C} hydroperoxide expected at 82.3 ppm and differing from that of δ_{C} a quaternary alcohol expected at 70.7 ppm.

Quorum Quenching Assays with Reporter Strains.

Fractions were tested for quorum quenching activity against all four agr types agr P3-YFP reporter strains AH1677 (type I), AH430 (type II), AH1747 (type III), and AH1872 (type IV). Sample stock solutions were dissolved in DMSO at a concentration of 2 mg/mL. Overnight cultures of reporter strains that were grown in TSB supplemented with chloramphenicol (Cam) were inoculated into fresh TSB containing Cam to achieve a working culture OD of 0.0006. Working culture was added to 96-well microtiter plates (Costar 3603) as was sample in order to achieve a final well volume of 200 μL. Mock vehicle (DMSO) treatments were included for each reporter strain. Microtiter plates were incubated at 37° C. with shaking (1250 rpm) in a Stuart SI505 incubator (Bibby Scientific, Burlington, N.J.) with a humidified chamber. Fluorescence (top reading, 493 nm excitation, 535 nm emission, gain 60) and optical density (OD) readings at 600 nm were recorded at 18 h using a Tecan Systems (San Jose, Calif.) Infinite M200 plate reader.

1. A pharmaceutical formulation comprising a compound having the following formula and a pharmaceutically acceptable excipient wherein the compound is 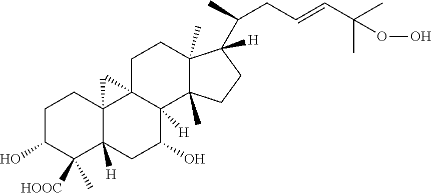 [SMILES: C[C@@H](C/C=C/C(C)(C)OO)[C@@H]1CC[C@]2(C)[C@H]3[C@H](O)C[C@@H]4[C@@](C)(C(=O)O)[C@H](O)CC[C@]45C[C@]35CC[C@@]12C]
or salt thereof.

2. The pharmaceutical formulation of claim 1 in the form of a particle, bead, tablet, capsule, or pill.

3. The pharmaceutical formulation of claim 1 in the form of a lotion, liquid, or gel.

4. The pharmaceutical formulation of claim 1 further comprising another antibiotic agent.

5. A method of treating or preventing a bacterial infection comprising administering to a subject with an effective amount of a pharmaceutical formula of claim 1.

6. The method of claim 5, wherein the subject is at risk of, exhibiting symptoms of, or diagnosed with toxic shock syndrome, scalded skin syndrome, abscesses, furuncles, cellulitis, folliculitis, bloodstream infections, medical device infections, pneumonia, osteomyelitis, staphylococcal food poisoning, skin and soft tissue infections, endocarditis, eczema, atopic dermatitis, psoriasis, impetigo, septic arthritis, brain abscess, burn wounds, venous ulcers, diabetic foot ulcers, surgical wounds, post-operation infections, carbuncles, meningitis, bacteremia, necrotizing pneumonia, or necrotizing fasciitis.

7. The method of claim 5, wherein administering is contacting the skin of subject with the pharmaceutical formulation.

8. The method of claim 5, wherein the pharmaceutical formula is administered in combination with an antibiotic agent.

9. A pharmaceutical formulation comprising a compound having the following formula and a pharmaceutically acceptable excipient wherein the compound is 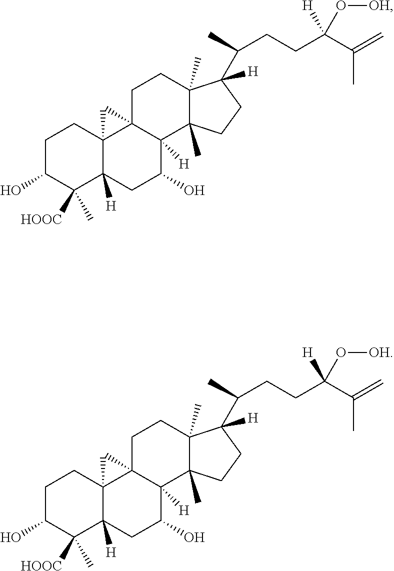 [SMILES: C=C(C)[C@@H](CC[C@H](C)[C@@H]1CC[C@]2(C)[C@H]3[C@H](O)C[C@@H]4[C@@](C)(C(=O)O)[C@H](O)CC[C@]45C[C@]35CC[C@@]12C)OO ; C=C(C)[C@H](CC[C@H](C)[C@@H]1CC[C@]2(C)[C@H]3[C@H](O)C[C@@H]4[C@@](C)(C(=O)O)[C@H](O)CC[C@]45C[C@]35CC[C@@]12C)OO]
or salt thereof.

10. The pharmaceutical formulation of claim 9 in the form of a particle, bead, tablet, capsule, or pill.

11. The pharmaceutical formulation of claim 9 in the form of a lotion, liquid, or gel.

12. The pharmaceutical formulation of claim 9 further comprising another antibiotic agent.

13. A method of treating or preventing a bacterial infection comprising administering to a subject with an effective amount of a pharmaceutical formula of claim 9.

14. The method of claim 13, wherein the subject is at risk of, exhibiting symptoms of, or diagnosed with toxic shock syndrome, scalded skin syndrome, abscesses, furuncles, cellulitis, folliculitis, bloodstream infections, medical device infections, pneumonia, osteomyelitis, staphylococcal food poisoning, skin and soft tissue infections, endocarditis, eczema, atopic dermatitis, psoriasis, impetigo, septic arthritis, brain abscess, burn wounds, venous ulcers, diabetic foot ulcers, surgical wounds, post-operation infections, carbuncles, meningitis, bacteremia, necrotizing pneumonia, or necrotizing fasciitis.

15. The method of claim 13, wherein administering is contacting the skin of subject with the pharmaceutical formulation.

16. The method of claim 13, wherein the pharmaceutical formula is administered in combination with an antibiotic agent.
